(19) United States
(12) Patent Application Publication
(10) Pub. No.: US 2023/0364277 A1
(43) Pub. Date: Nov. 16, 2023

(54) E-SELECTIN TARGETING AGENTS

(71) Applicant: GLYCOMIMETICS, INC., Rockville, MD (US)

(72) Inventors: John L. MAGNANI, Gaithersburg, MD (US); John M. PETERSON, Slate Hill, NY (US); William E. FOGLER, Lutherville-Timonium, MD (US); Myung-Gi BAEK, Clarksburg, MD (US)

(73) Assignee: GLYCOMIMETICS, INC., Rockville, MD (US)

(21) Appl. No.: 18/245,574

(22) PCT Filed: Sep. 17, 2021
(86) PCT No.: PCT/US2021/050955

§ 371 Date: Mar. 16, 2023

Related U.S. Application Data
(60) Provisional application No. 63/079,997, filed on Sep. 17, 2020.

(51) Int. Cl. A61K 51/12; A61K 51/04

(52) U.S. Cl. CPC A61K 51/1244; A61K 51/0491; A61K 2123/00

(57) ABSTRACT

E-selectin ligands which are useful for the synthesis of E-selectin ligand-bearing carriers, wherein said E-selectin ligand-bearing carriers are directly or indirectly linked to or associated with at least one therapeutic agent, diagnostic agent, imaging agent, or radiopharmaceutical are described herein.

This application claims the benefit of priority of U.S. Provisional Application No. 63/079,997, filed Sep. 17, 2020, the contents of which are herein incorporated by reference in their entirety.

E-selectin ligands which are useful for the synthesis of E-selectin ligand-bearing carriers, wherein said E-selectin ligand-bearing carriers are directly or indirectly linked to or associated with at least one therapeutic agent, diagnostic agent, imaging agent, or radiopharmaceutical, and the use of said carriers for treating and/or preventing a disease, disorder, or condition, or for diagnosing or imaging E-selectin expressing tissues are described herein.

The selectins are a family of cell surface adhesion proteins (E, P, and L-selectins) that bind interacting cell surfaces through calcium-dependent recognition of specific carbohydrates sequences expressing the sialyl Le^{x/a} epitope. P and L-selectin require the additional interaction of sulfate groups for tight binding whereas E-selectin does not. The capital letters refer to the cell-types that express each selectin. L-selectin is found on leukocytes and functions mainly in cell trafficking. P-selectin is found on platelets but is also known to be in preformed vesicles inside endothelial cells. E-selectin is expressed exclusively on the surface of endothelial cells and plays a major role in the initial steps of the inflammatory response, but is also involved in the formation of new blood vessels during angiogenesis, as well as binding to cancer cells in the vascular protective niches of the bone marrow.

In contrast to P and L-selectins, E-selectin usually requires de novo protein synthesis after chemokine activation (i.e. TNF-α, IL1-β) and is present on inflammatory endothelium but not usually on the endothelium of the normal vasculature. Exceptions include areas of active propagation of the endothelium during angiogenesis as well as in the bone marrow and skin. Given this specificity and its ability to bind a unique carbohydrate epitope, the expression of E-selectin can be used for targeting diseases such as inflammation and cancer. Targeting molecules can include, for example, antibodies, aptamers, peptides, carbohydrates, or mimics of these molecules, such as glycomimetics or peptidomimetics. Such targeting agents can be conjugated with or direct a “payload molecule”, such as a drug or imaging agent to either treat or diagnose and localize the disease.

Successful growth and progression of cancer requires vascularization of the tumor to exchange oxygen and metabolites required for cell proliferation. This process known as angiogenesis is initiated by activated endothelial progenitors cells that highly express E-selectin (Oh et. al., Blood 110(12):3891-3899, 2007). Blocking angiogenesis is one of the major strategies investigated for the development of novel therapeutics for cancer. Yasuda et al (Am. J. Physiol. Cell Physiol. 282:C917-C925, 2002) showed that E-selectin functions in the initial formation of tubelike structures among the activated endothelial progenitor cells that forms the basis of neovascularization and angiogenesis. Liposomes are a class of nanoparticles that have long been used to encapsulate drugs for increased efficacy. E. L. Vodovozova (Eur. J Cancer, 36:942-949, 2000) targeted liposomes carrying the drug melphalan to E-selectin by chemically linking the E-selectin carbohydrate ligand, sialyl Lex to lipids incorporated in the liposomes. These drug-filled liposomes expressing surface sialyl Lex targeted E-selectin and significantly enhanced survival in a mouse breast cancer model with a high incidence of spontaneous mammary adenocarcinoma. The localization of these targeted drug-carrying liposomes was further investigated using fluorescently labeled liposomes with specific cell type markers. The targeted sialyl Lex expressing liposomes co-localized with the CD31 marker of the vascular endothelium as determined by confocal microscopy (Kuznetsova et. al., J. Drug Target. 22(3):242-250, 2014). These studies demonstrate that targeting E-selectin expressed in the tumor microvasculature with drug-carrying nanoparticles (liposomes) is an effective strategy to enhance the efficacy of chemotherapy.

Biopsies from breast cancer patients were analyzed for expression of specific markers for targeting. E-selectin expression was found to be elevated in the angiogenic vasculature of malignant breast cancer biopsies compared with their benign counterparts (23.86% vs 2.47%; p=0.0005). In fact, E-selectin was further found to be significantly increased in estrogen-receptor negative tumors compared with estrogen-negative ones (p=0.005) (Nguyen et. al., Am J. Pathology 150(4):1307-1314, 1997). E-selectin in the breast cancer tumor vasculature was targeted using an aptamer directed against E-selectin (Mann et. al., PLoS ONE 5(9):e13050, 2010). This aptamer was chemically conjugated to liposomes and was shown to directly target these liposomes to the tumor vasculature of breast cancer xenografts in nude mice (Mann et. al., Oncotarget 2:298-304, 2011).

The expression of tumor markers was screened using the Eos Hu03 GeneChip array. The results of this analysis showed that E-selectin mRNA is uniquely overexpressed in prostate cancer epithelium. Antibodies directed against E-selectin supported these findings using immunohistochemistry. An antibody drug conjugate (ADC) was created by chemically linking auristatin-E to the E-selectin directed antibody. Efficacy of targeting E-selectin was demonstrated however only on endothelial cells expressing E-selectin (Bhaskar et. al., Cancer Research 63:6387-6394, 2003).

E-selectin functions in the initiating of an inflammatory response and it is specifically expressed by de novo protein synthesis only after stimulation by inflammatory chemokines. Thus, E-selectin has been targeted not only for cancer therapy, but also for several different inflammatory disorders found in disorders such as cardiovascular disease, diabetes, and inflammatory bowel disease. For example, liposomes targeted to E-selectin on activated vasculature endothelial cells by conjugating an anti-E-selectin antibody to the liposome were used to deliver doxorubicin to E-selectin expressing endothelial cells (Spragg et. al., Proc. Natl. Acad. Sci. 94:8795-8800, 1997). In another study, a conjugate of a peptide directed to E-selectin with dexamethasone was constructed to target the anti-inflammatory drug to aortic atherosclerotic lesions (Tsoref et. al., J of Controlled Release 288:136-147, 2018).

Besides specifically delivering drugs as novel therapies for cancers and inflammatory disorders, E-selectin can also be used as a target for imaging and localization of inflammatory events or the tumor vasculature. A review covering such nanoparticles for the imaging of inflammation identifies E-selectin as a target (Jin et al., Acta Pharmaceutica Sinica B 8(1):23-33, 2018). Imaging agents such as technetium 99 m have been used to conjugate with an anti-E-selectin Fab fragment to specifically image synovitis in rheumatoid arthritis (Jamar F. et. al., Rheumatology 41:53-61, 2002). Other imaging agents such as cross-linked iron oxide superparamagnetic nanoparticles have been conjugated with anti-E-selectin Fab fragments to image areas of inflammation and angiogenesis by magnetic imaging resonance (MRI), (Kang et al., Bioconjugate Chem. 13:122-127, 2002).

The present disclosure is directed to E-selectin ligands which are useful for the synthesis of E-selectin ligand-bearing carriers. These E-selectin ligand-bearing carriers are capable of being directly or indirectly linked to or associated with at least one therapeutic agent, diagnostic agent, imaging agent, or radiopharmaceutical.

Disclosed are compounds of Formula (I):

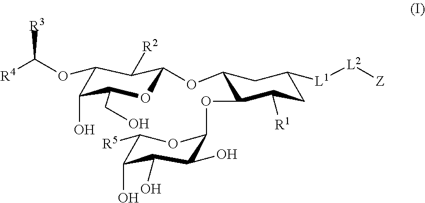

and pharmaceutically acceptable salts thereof, wherein R¹, R², R³, R⁴, R⁵, L¹, L², and Z are as defined herein.

In some embodiments, a process for making E-selectin ligand-bearing carriers of Formula (II) is provided, wherein the process comprises reacting or associating a compound of Formula (I), or a protected form thereof, with a compound of Formula (III):

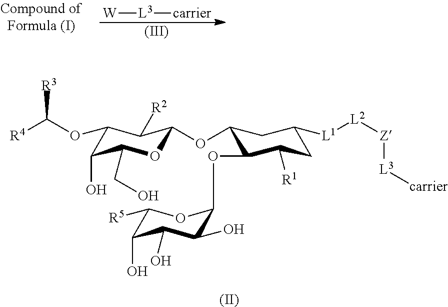

wherein R¹, R², R³, R⁴, R⁵, L¹, L², L³, Z′, W, and carrier are as defined herein.

In some embodiments, a method for treatment and/or prevention of at least one disease, disorder, or condition is disclosed, the method comprising administering to a subject in need thereof at least one compound of Formula (II) or a composition comprising the same, wherein said at least one compound of Formula (II) delivers an effective amount of at least one therapeutic agent.

In some embodiments, a method for treatment and/or prevention of at least one disease, disorder, or condition is disclosed, the method comprising administering to a subject in need thereof at least one compound of Formula (II) or a composition comprising the same, wherein said at least one compound of Formula (II) delivers an effective amount of at least one radiopharmaceutical. In some embodiments, the at least one radiopharmaceutical is ¹⁷⁷Lu. In some embodiments, the at least one radiopharmaceutical is ¹¹¹In.

In some embodiments, a method for diagnosing at least one disease, disorder, or condition is disclosed, the method comprising administering to a subject in need thereof an effective amount of at least one compound of Formula (II) or a composition comprising the same, wherein at least one diagnostic agent is directly or indirectly linked to or associated with said at least one compound of Formula (II).

In some embodiments, a method for diagnosing or imaging E-selectin expressing tissues is disclosed, the method comprising administering to a subject in need thereof an effective amount of at least one compound of Formula (II) or a composition comprising the same, wherein at least one diagnostic agent or at least one imaging agent is directly or indirectly linked to or associated with said at least one compound of Formula (II).

In some embodiments, the at least one imaging agent is Gd³⁺. In some embodiments, the imaging agent is technetium 99 m. In some embodiments, the at least one imaging agent is chosen from cross-linked iron oxide superparamagnetic nanoparticles. In some embodiments, the at least one imaging agent is capable of imaging blood vessels. In some embodiments, the at least one imaging agent is capable of imaging angiogenesis.

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description and attached drawings.

### BRIEF DESCRIPTION OF THE DRAWINGS

FIG. 1 is a diagram illustrating the synthesis of Compound 3.

FIG. 2 is a diagram illustrating the prophetic synthesis of Compound 5.

FIG. 3 is a diagram illustrating the prophetic synthesis of Compound 11.

FIG. 4 is a diagram illustrating the prophetic synthesis of Compound 13.

FIG. 5 is a diagram illustrating interaction of E-selectin-Fc chimera with captured liposomes containing 5% Compound 3 (referred to as compound A).

FIG. 6 is a diagram illustrating total binding of Compound 3 (referred to as compound A) liposomes to immobilized E-selectin or BSA.

FIG. 7 is a diagram illustrating induction of E-selectin expression on HUVEC by TNFα.

FIG. 8 is a diagram illustrating liposomes containing Compound 3 (referred to as compound A) binding to activated HUVEC.

FIG. 9 is a diagram illustrating exemplary conjugation chemistry useful for the synthesis of compounds of Formula (II).

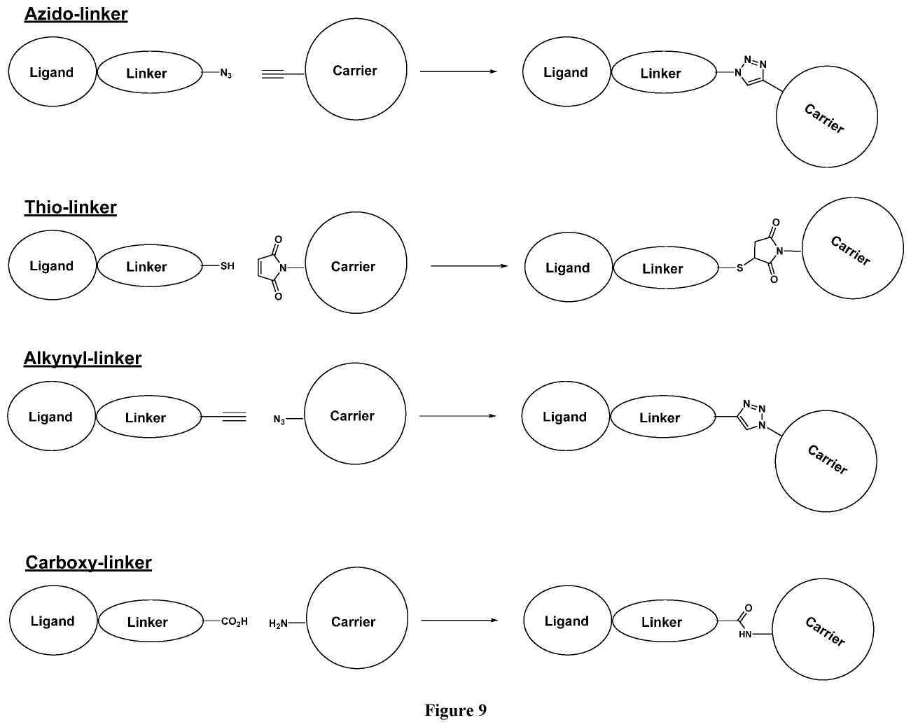

FIG. 10 is a series of representative images showing the kinetics of biodistribution of IRdye780-labeled liposomes comprising Compound 3 by fluorescence imaging (FLI) following treatment of female C.B.-17 SCID mice with established A549 human NSCLC tumors, where the numbers in the upper left corner of each image is the mean FLI signal of each group (N=3 mice/time point).

Disclosed herein are E-selectin ligands, which are useful for the synthesis of E-selectin ligand-bearing carriers. The E-selectin ligand-bearing carriers may be useful for targeting therapeutic agents to E-selectin expressing vascular compartments of tissues for treating diseases, disorders, or conditions, including cancer, inflammatory diseases, and/or angiogenesis. The E-selectin ligand-bearing carriers may also be useful for imaging E-selectin expressing vascular components of tissues to by linking or associating an E-selectin ligand-bearing carrier of the present disclosure with at least one imaging agent, such as an MRI contrast agent.

In some embodiments, disclosed are compounds of Formula (I):

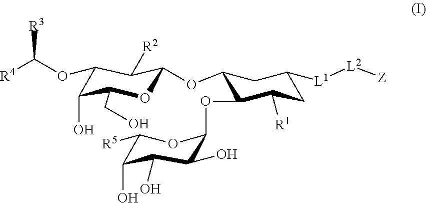

and pharmaceutically acceptable salts thereof, wherein
R¹ is chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl,

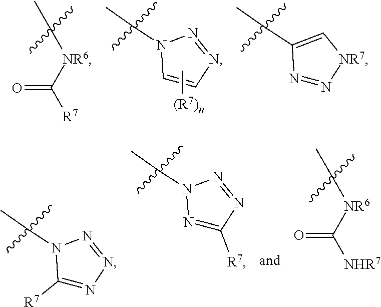

groups, wherein n is chosen from integers ranging from 0 to 2, R⁶ is chosen from H, C₁₋₁₂ alkyl, C₂₋₁₂ alkenyl, C₂₋₁₂ alkynyl, C₄₋₁₆ cycloalkylalkyl, and —C(═O)R⁷ groups, and each R⁷ is independently chosen from H, C₁₋₁₂ alkyl, C₂₋₁₂ alkenyl, C₂₋₁₂ alkynyl, C₄₋₁₆ cycloalkylalkyl, C₆₋₁₈ aryl, and C₁₋₁₃ heteroaryl groups;
R² is chosen from —OH, —OY¹, halo, —NH₂, —NHY¹, —NY¹Y², —OC(═O)Y¹, —NHC(═O)Y¹, —NHC(═O)NHY¹, and —NHC(═O)NY¹Y² groups, wherein Y¹ and Y², which may be the same or different, are independently chosen from C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₄₋₁₆ cycloalkylalkyl, C₂₋₁₂ heterocyclyl, C₆₋₁₈ aryl, and C₁₋₁₃ heteroaryl groups, or Y¹ and Y² may join together along with the heteroatom to which they are attached to form an optionally substituted, saturated or unsaturated ring;
R³ is chosen from —CN, —CH₂CN, —C(═O)Y³, —C(═O)OH, —C(═O)OY³, —C(═O)NH₂, —C(═O)NHOH, —C(═O)NHOCH₃, —NHCN, —C(═O)NHY³, —C(═O)NY³Y⁴, —S(═O)₂Y³, —S(═O)₂OY³, —S(═O)₂NH₂, —S(═O)₂NHY³, and —S(═O)₂NY³Y⁴ groups, wherein Y³ and Y⁴, which may be identical or different, are independently chosen from C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₁₋₁₃ heterocyclyl, and C₇₋₁₂ arylalkyl groups, or Y³ and Y⁴ join together along with the heteroatom to which they are attached to form an optionally substituted, saturated or unsaturated ring;
R⁴ is chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₁₋₁₂ alkoxy, C₄₋₁₆ cycloalkylalkyl, C₆₋₁₈ aryl, and C₂₋₁₃ heteroaryl groups;
R⁵ is chosen from —CN, C₁₋₁₂ alkyl, and C₁₋₁₂ haloalkyl groups;
L¹ is chosen from

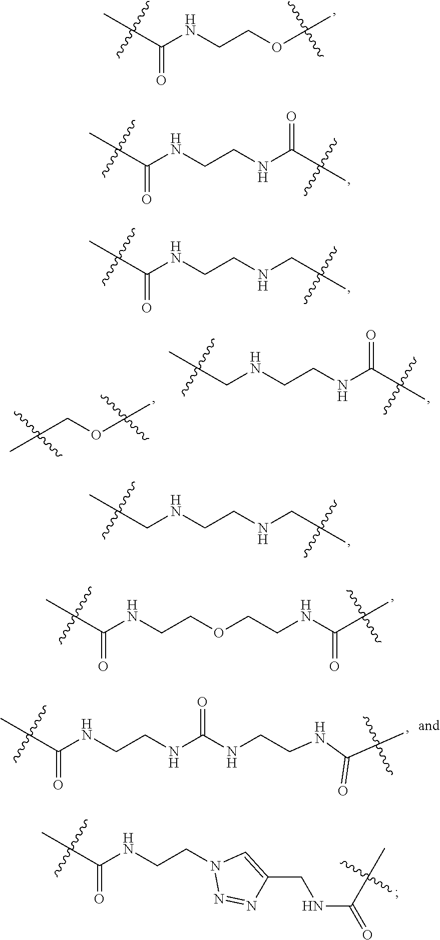

L² is chosen from —CH₂(OCH₂CH₂)ₘV—, —CH₂CH₂(OCH₂CH₂)ₘV—, and —(CH₂)ₘV— groups, wherein V is chosen from a bond, —O—, —OCH₂—, —NHC(═O)CH₂CH₂—, and —C(═O)NHCH₂CH₂—, and wherein m is chosen from integers ranging from 1 to 46; and
Z is chosen from lipids, nucleophiles, electrophiles, and groups capable of undergoing a cycloaddition reaction.

In some embodiments, R¹ is chosen from H, C₁₋₄ alkyl, and C₁₋₄ haloalkyl groups. In some embodiments, R¹ is chosen from H, methyl, ethyl, —CH₂F, —CHF₂, —CF₃, —CH₂CH₂F, —CH₂CHF₂, and —CH₂CF₃. In some embodiments, R¹ is H. In some embodiments, R¹ is chosen from methyl and ethyl. In some embodiments, R¹ is methyl. In some embodiments, R¹ is ethyl.

In some embodiments, R¹ is chosen from

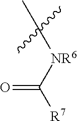

groups.

In some embodiments, R¹ is chosen from

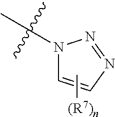

groups.

In some embodiments, R¹ is chosen from

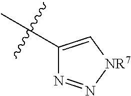

groups.

In some embodiments, R¹ is chosen from

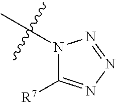

groups.

In some embodiments, R¹ is chosen from

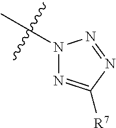

groups.

In some embodiments, R¹ is chosen from

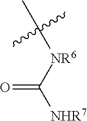

groups.

In some embodiments, R⁶ is chosen from H, C₁₋₈ alkyl, and —C(═O)R⁷ groups. In some embodiments, R⁶ is chosen from H and C₁₋₈ alkyl groups. In some embodiments, R⁶ is chosen from C₁₋₄ alkyl groups. In some embodiments, R⁶ is H.

In some embodiments, each R⁷ is independently chosen from H, C₁₋₈ alkyl, C₆₋₁₈ aryl groups, and C₁₋₁₃ heteroaryl groups. In some embodiments, at least one R⁷ is chosen from C₁₋₈ alkyl groups. In some embodiments, at least one R⁷ is chosen from C₁₋₄ alkyl groups. In some embodiments, at least one R⁷ is chosen from methyl and ethyl. In some embodiments, at least one R⁷ is H. In some embodiments, at least one R⁷ is methyl. In some embodiments, at least one R⁷ is ethyl.

In some embodiments, at least one R⁷ is chosen from

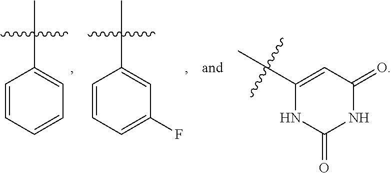

In some embodiments, R¹ is

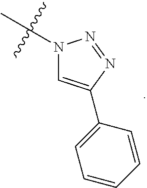 [SMILES: *n1cc(-c2ccccc2)nn1]

In some embodiments, R¹ is

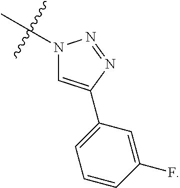 [SMILES: *n1cc(-c2cccc(F)c2)nn1]

In some embodiments, R¹ is

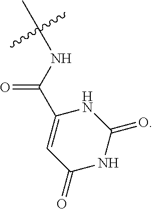 [SMILES: *NC(=O)c1cc(=O)[nH]c(=O)[nH]1]

In some embodiments, R¹ is

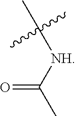 [SMILES: *NC(C)=O]

In some embodiments, R² is chosen from —OH, —OY¹, —OC(═O)Y¹, and —NHC(═O)Y¹ groups, wherein Y¹ is chosen from C₁₋₈ alkyl, C₄₋₁₆ cycloalkylalkyl, C₂₋₁₂ heterocyclyl, C₆₋₁₈ aryl, and C₁₋₁₃ heteroaryl groups. In some embodiments, R² is chosen from —OY¹ groups. In some embodiments, R² is chosen from —OC(═O)Y¹ groups. In some embodiments, R² is chosen from —NHC(═O)Y¹ groups. In some embodiments, R₂ is —OH.

In some embodiments, R² is chosen from

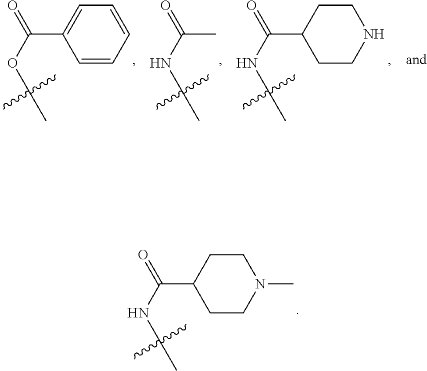

In some embodiments, R² is

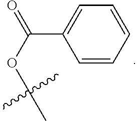 [SMILES: *OC(=O)c1ccccc1]

In some embodiments, R² is

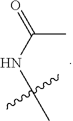 [SMILES: *NC(C)=O]

In some embodiments, R³ is chosen from —C(═O)OH, —C(═O)OY³, —C(═O)NH₂, —C(═O)NHOH, —C(═O)NHOCH₃, —C(═O)NHY³, and —C(═O)NY³Y⁴ groups. In some embodiments, R³ is chosen from —C(═O)OH, —C(═O)NHY³, and —C(═O)NY³Y⁴ groups. In some embodiments, Y³ and Y⁴, which may be identical or different, are independently chosen from C₁₋₈ alkyl, C₁₋₈ haloalkyl, and C₇₋₁₂ arylalkyl groups. In some embodiments, Y³ and Y⁴ join together along with the heteroatom to which they are attached to form an optionally substituted, saturated or unsaturated ring.

In some embodiments, R³ is chosen from

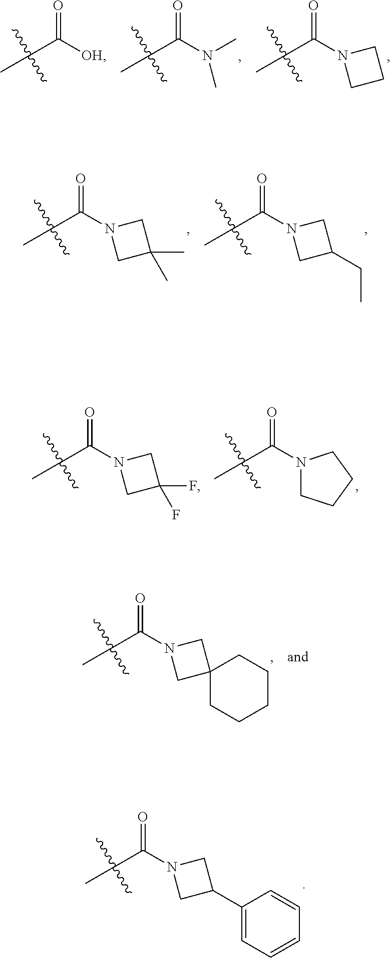

In some embodiments, R³ is chosen from

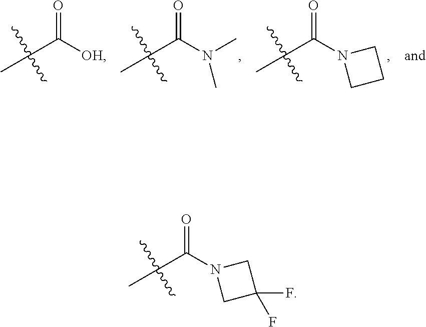

In some embodiments, R³ is

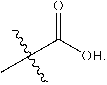 [SMILES: *C(=O)O]

In some embodiments, R³ is

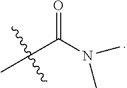 [SMILES: *C(=O)N(C)C]

In some embodiments, R³ is

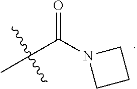 [SMILES: *C(=O)N1CCC1]

In some embodiments, R³ is

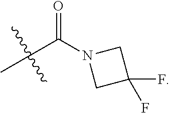 [SMILES: *C(=O)N1CC(F)(F)C1]

In some embodiments, R⁴ is chosen from C₁₋₈ alkyl groups. In some embodiments, R⁴ is chosen from C₁₋₈ haloalkyl groups. In some embodiments, R⁴ is chosen from C₄₋₁₆ cycloalkylalkyl groups. In some embodiments, R⁴ is chosen from C₄₋₈ cycloalkylalkyl groups. In some embodiments, R⁴ is chosen from propyl, cyclopropylmethyl, and cyclohexylmethyl. In some embodiments, R⁴ is propyl. In some embodiments, R⁴ is cyclopropylmethyl. In some embodiments, R⁴ is cyclohexylmethyl.

In some embodiments, R⁵ is chosen from halomethyl groups. In some embodiments, R⁵ is CF₃. In some embodiments, R⁵ is CH₃. In some embodiments, R⁵ is CN.

In some embodiments, L¹ is

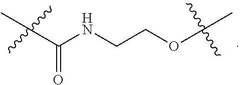 [SMILES: *OCCNC(*)=O]

In some embodiments, L¹ is

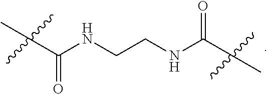 [SMILES: *C(=O)NCCNC(*)=O]

In some embodiments, L¹ is

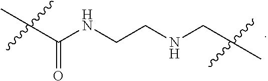 [SMILES: *CNCCNC(*)=O]

In some embodiments, L¹ is

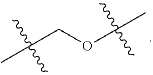 [SMILES: *CO*]

In some embodiments, L¹ is

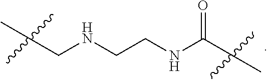 [SMILES: *CNCCNC(*)=O]

In some embodiments, L¹ is

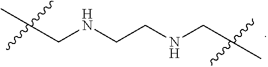 [SMILES: *CNCCNC*]

In some embodiments, L¹ is

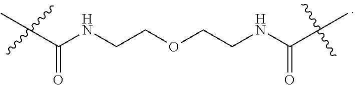 [SMILES: *C(=O)NCCOCCNC(*)=O]

In some embodiments, L¹ is

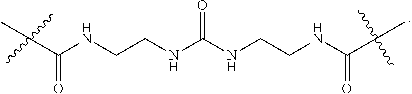 [SMILES: *C(=O)NCCNC(=O)NCCNC(*)=O]

In some embodiments, L¹ is

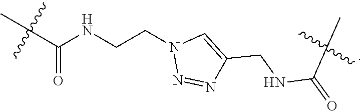 [SMILES: *C(=O)NCCn1cc(CNC(*)=O)nn1]

In some embodiments, L² is chosen from —(CH₂)ₘV— groups. In some embodiments, L² is chosen from —(CH₂)ₘV— groups, wherein V is —C(═O)NHCH₂CH₂—. In some embodiments, L² is chosen from —(CH₂)ₘV— groups, wherein V is —C(═O)NHCH₂CH₂—, and m is chosen from integers ranging from 1 to 30. In some embodiments, m is chosen from integers ranging from 1 to 20. In some embodiments, m is chosen from integers ranging from 1 to 10. In some embodiments, m is chosen from integers ranging from 1 to 5. In some embodiments, m is 5. In some embodiments, m is 4. In some embodiments, m is 3. In some embodiments, m is 2. In some embodiments, m is 1.

In some embodiments, L² is —CH₂C(═O)NHCH₂CH₂—. In some embodiments, L² is —CH₂CH₂C(═O)NHCH₂CH₂—. In some embodiments, L² is —CH₂CH₂CH₂C(═O)NHCH₂CH₂—. In some embodiments, L² is —CH₂CH₂CH₂CH₂C(═O)NHCH₂CH₂—.

In some embodiments, L² is chosen from —CH₂(OCH₂CH₂)ₘV— groups. In some embodiments, L² is chosen from —CH₂(OCH₂CH₂)ₘV— groups, wherein V is chosen from a bond, —O—, and —OCH₂—. In some embodiments, L² is chosen from —CH₂(OCH₂CH₂)ₘV— groups, wherein V is chosen from a bond, —O—, and —OCH₂—, and m is chosen from integers ranging from 1 to 30. In some embodiments, m is chosen from integers ranging from 1-20. In some embodiments, m is chosen from integers ranging from 1-10. In some embodiments, m is chosen from integers ranging from 1-6. In some embodiments, m is 6. In some embodiments, m is 5. In some embodiments, m is 4. In some embodiments, m is 3. In some embodiments, m is 2. In some embodiments, m is 1.

In some embodiments, L² is —CH₂OCH₂CH₂—. In some embodiments, L² is —CH₂(OCH₂CH₂)₂—. In some embodiments, L² is —CH₂(OCH₂CH₂)₄—. In some embodiments, L² is —CH₂(OCH₂CH₂)₆—. In some embodiments, L² is —CH₂OCH₂CH₂O—. In some embodiments, L² is —CH₂(OCH₂CH₂)₂O—. In some embodiments, L² is —CH₂(OCH₂CH₂)₄O—. In some embodiments, L² is —CH₂(OCH₂CH₂)₆O—. In some embodiments, L² is —CH₂OCH₂CH₂OCH₂—. In some embodiments, L² is —CH₂(OCH₂CH₂)₂OCH₂—. In some embodiments, L² is —CH₂(OCH₂CH₂)₄OCH₂—. In some embodiments, L² is —CH₂(OCH₂CH₂)₆OCH₂—.

In some embodiments, L² is chosen from —CH₂CH₂(OCH₂CH₂)ₘV— groups. In some embodiments, L² is chosen from —CH₂CH₂(OCH₂CH₂)ₘV— groups, wherein V is chosen from a bond, —O—, —OCH₂—, and —NHC(═O)CH₂CH₂—. In some embodiments, L² is chosen from —CH₂CH₂(OCH₂CH₂)ₘV— groups, wherein V is chosen from a bond, —O—, —OCH₂—, and —NHC(═O)CH₂CH₂—, and m is chosen from integers ranging from 1 to 30. In some embodiments, m is chosen from integers ranging from 1 to 20. In some embodiments, m is chosen from integers ranging from 1 to 14. In some embodiments, m is 12. In some embodiments, m is 10. In some embodiments, m is 8. In some embodiments, m is 6. In some embodiments, m is 4. In some embodiments, m is 3. In some embodiments, m is 2. In some embodiments, m is 1.

In some embodiments, L² is —CH₂CH₂OCH₂CH₂—. In some embodiments, L² is —CH₂CH₂(OCH₂CH₂)₂—. In some embodiments, L² is —CH₂CH₂(OCH₂CH₂)₃—. In some embodiments, L² is —CH₂CH₂(OCH₂CH₂)₄—. In some embodiments, L² is —CH₂CH₂(OCH₂CH₂)₆—. In some embodiments, L² is —CH₂CH₂(OCH₂CH₂)₈—. In some embodiments, L² is —CH₂CH₂(OCH₂CH₂)₁₀—. In some embodiments, L² is —CH₂CH₂(OCH₂CH₂)₁₂—.

In some embodiments, L² is —CH₂CH₂OCH₂CH₂O—. In some embodiments, L² is —CH₂CH₂(OCH₂CH₂)₂O—. In some embodiments, L² is —CH₂CH₂(OCH₂CH₂)₃O—. In some embodiments, L² is —CH₂CH₂(OCH₂CH₂)₄O—. In some embodiments, L² is —CH₂CH₂(OCH₂CH₂)₆O—. In some embodiments, L² is —CH₂CH₂(OCH₂CH₂)₈O—. In some embodiments, L² is —CH₂CH₂(OCH₂CH₂)₁₀O—. In some embodiments, L² is —CH₂CH₂(OCH₂CH₂)₁₂O—.

In some embodiments, L² is —CH₂CH₂OCH₂CH₂OCH₃. In some embodiments, L² is —CH₂CH₂(OCH₂CH₂)₂OCH₃—. In some embodiments, L² is —CH₂CH₂(OCH₂CH₂)₃OCH₃—. In some embodiments, L² is —CH₂CH₂(OCH₂CH₂)₄OCH₃—. In some embodiments, L² is —CH₂CH₂(OCH₂CH₂)₆OCH₃—. In some embodiments, L² is —CH₂CH₂(OCH₂CH₂)₈OCH₃—. In some embodiments, L² is —CH₂CH₂(OCH₂CH₂)₁₀OCH₃—. In some embodiments, L² is —CH₂CH₂(OCH₂CH₂)₁₂OCH₃—.

In some embodiments, L² is —CH₂CH₂OCH₂CH₂NH(═O)CH₂CH₂—. In some embodiments, L² is —CH₂CH₂(OCH₂CH₂)₂NH(═O)CH₂CH₂—. In some embodiments, L² is —CH₂CH₂(OCH₂CH₂)₃NH(═O)CH₂CH₂—. In some embodiments, L² is —CH₂CH₂(OCH₂CH₂)₄NH(═O)CH₂CH₂—. In some embodiments, L² is —CH₂CH₂(OCH₂CH₂)₆NH(═O)CH₂CH₂—. In some embodiments, L² is —CH₂CH₂(OCH₂CH₂)₈NH(═O)CH₂CH₂—. In some embodiments, L² is —CH₂CH₂(OCH₂CH₂)₁₀NH(═O)CH₂CH₂—. In some embodiments, L² is —CH₂CH₂(OCH₂CH₂)₁₂NH(═O)CH₂CH₂—.

In some embodiments, Z is chosen from lipids. In some embodiments, Z is chosen from glycerol-based lipids. In some embodiments, Z is chosen from phospholipids. In some embodiments, Z is chosen from symmetric phospholipids. In some embodiments, Z is chosen from asymmetric phospholipids. In some embodiments, Z is chosen from sphingolipids. In some embodiments, Z is chosen from ceramides.

In some embodiments, Z is chosen from phospholipids that have 16 to 30 carbon atoms. In some embodiments, Z is chosen from phospholipids that have 16, 20, 22, 24, 26, 28, or 30 carbon atoms. In some embodiments, Z is chosen from phospholipids that have 16 carbon atoms. In some embodiments, Z is chosen from phospholipids that have 18 carbon atoms. In some embodiments, Z is chosen from phospholipids that have 20 carbon atoms. In some embodiments, Z is chosen from phospholipids that have 22 carbon atoms. In some embodiments, Z is chosen from phospholipids that have 24 carbon atoms. In some embodiments, Z is chosen from phospholipids that have 26 carbon atoms. In some embodiments, Z is chosen from phospholipids that have 28 carbon atoms. In some embodiments, Z is chosen from phospholipids that have 30 carbon atoms.

In some embodiments, Z is chosen from saturated phospholipids. In some embodiments, Z is chosen from unsaturated phospholipids. In some embodiments, Z is chosen from phosphatidylcholine, phosphatidyethanolamine, phosphatidic acid, and phosphatidyl inositol. In some embodiments, Z is chosen from phospholipid derivatives of 6-cis-octadecenoic, 9-cis-octadecenoic, 9-trans-octadecenoic, 9-cis-12-octadecadienoic, 9-cis-12-cis-15-cis-octadecatrienoic, 11-cis-eicosenoic, 5, 8, 11, 14 (all-cis) eicosatetraenoic, 13-cis-docosenoic, 4, 7, 10, 13, 16, 19 (all-cis) docosahexaenoic, and/or 15-cis-tetracosenoic acids. In some embodiments, Z is chosen from phospholipid derivatives of stearoyl, oleoyl, linoleoyl, arachidonoyl, and/or docosahexaenoyl acids.

In some embodiments, the phospholipid is

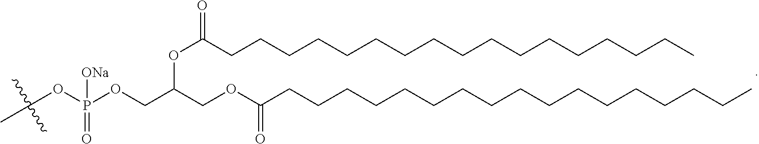

In some embodiments, Z is chosen from nucleophiles. In some embodiments, the nucleophiles are chosen from amines, mercaptans, and alcohols. In some embodiments, Z is chosen from amine groups.

In some embodiments, Z is chosen from electrophiles. In some embodiments, the electrophiles are chosen from epoxides, aziridines, episulfides, sulfates, sulfonates, carbonates, lactones, lactams, halides, acid halides, and esters. In some embodiments, the electrophiles are chosen from epoxides. In some embodiments, the electrophiles are chosen from aziridines. In some embodiments, the electrophiles are chosen from episulfides. In some embodiments, the electrophiles are chosen from sulfates. In some embodiments, the electrophiles are chosen from sulfonates. In some embodiments, the electrophiles are chosen from carbonates. In some embodiments, the electrophiles are chosen from lactones. In some embodiments, the electrophiles are chosen from lactams. In some embodiments, the electrophiles are chosen from halides. In some embodiments, the electrophiles are chosen from acid halides. In some embodiments, the electrophiles are chosen from esters. In some embodiments, the esters are chosen from activated esters.

In some embodiments, Z is chosen from esters. In some embodiments, the esters are chosen from anhydrides, carboxylic acid-succinimides, carboxylic acid phosphoesters, and carboxylic acid imidazolides. In some embodiments, Z is chosen from esters formed with t-butanol, p-nitrophenol, 2,4-dinitrophenol, trichlorophenol, 1-hydroxy-1H-benzotriazole, 1-hydroxy-6-chloro-1H-benzotriazole, and N-hydroxysuccinimide.

In some embodiments, Z is chosen from —C(═O)Y⁶ groups, wherein Y⁶ is chosen from H, —OH, —O^{t}Bu, C₂₋₈ alkenyl, C₁₋₈ haloalkyl, C₁₋₈ alkoxy, halo, C₆₋₁₈ aryloxy, C₁₋₁₃ heteroaryloxy, and C₂₋₁₂ heterocyclyloxy groups and wherein the C₁₋₈ alkoxy, C₆₋₁₈ aryloxy, C₁₋₁₃ heteroaryloxy, and C₂₋₁₂ heterocyclyloxy groups are optionally substituted with at least one halo group.

In some embodiments, Z is chosen from groups capable of undergoing a cycloaddition reaction. In some embodiments, the groups capable of undergoing a cycloaddition reaction are chosen from alkenes, alkynes, dienes, dienophiles, 1,3-dipoles, and 1,3-dipolarophiles.

In some embodiments, the dienophiles are chosen from —C(═O)Y⁷ groups, wherein Y⁷ is chosen from alkene and alkyne groups, wherein said alkene and alkyne groups are optionally substituted with C₁₋₈ alkoxy, —C(═O)H, —C(═O)OY⁸, and —C(═O)NY⁸Y⁹ groups, wherein Y⁸ and Y⁹, which may be identical or different, are independently chosen from H and C₁₋₈ alkyl groups, or Y⁸ and Y⁹ join together along with the nitrogen to which they are attached to form an optionally substituted, saturated or unsaturated, 3-10 membered ring.

In some embodiments, the 1,3-dipoles are chosen from nitrile ylides, nitrile imines, nitrile oxides, diazolalkanes, azides, azomethine ylides, azomethine imines, nitrones, carbonyl ylides, carbonyl imines, and carbonyl oxides.

In some embodiments, Z is chosen from alkene and alkyne groups. In some embodiments, Z is chosen from alkene groups. In some embodiments, Z is chosen from alkyne groups. In some embodiments, Z is —CH═CH₂. In some embodiments, Z is —C≡CH.

In some embodiments, Z is chosen from dienes and dienophiles. In some embodiments, Z is chosen from dienes. In some embodiments, Z is chosen from dienophiles.

In some embodiments, Z is chosen from 1,3-dipoles and 1,3-dipolarophiles. In some embodiments, Z is chosen from 1,3-dipoles. In some embodiments, Z is chosen from 1,3-dipolarophiles.

In some embodiments, Z is chosen from —N₃, —NH₂, —N(OH)H, —SH, —OH, —Cl, —Br, —I, —CH═CH₂, —C≡CH,

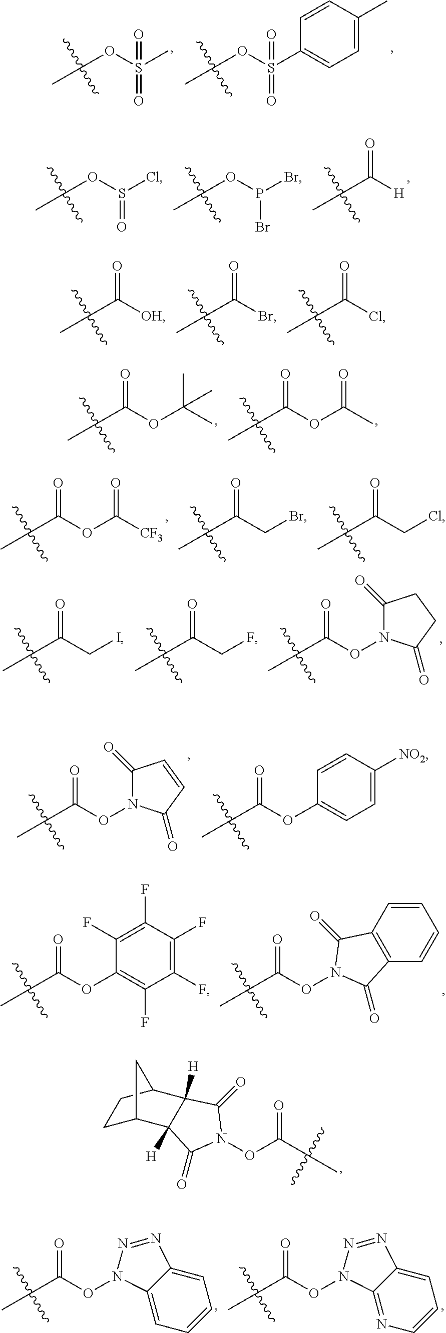

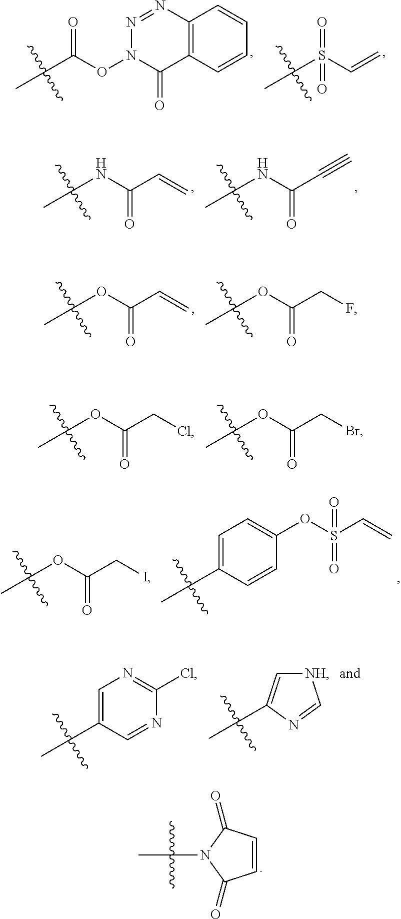

In some embodiments, Z is —O—NH₂.

In some embodiments, Z is —N₃.

In some embodiments, Z is chosen from —NH₂, —SH, and —OH. In some embodiments, Z is —NH₂. In some embodiments, Z is —SH. In some embodiments, Z is —OH.

In some embodiments, Z is —C(═O)H. In some embodiments, Z is —C(═O)OH. In some embodiments, Z is —C(═O)Cl. In some embodiments, Z is —C(═O)O^{t}Bu.

In some embodiments, the compound of Formula (I) is chosen from:

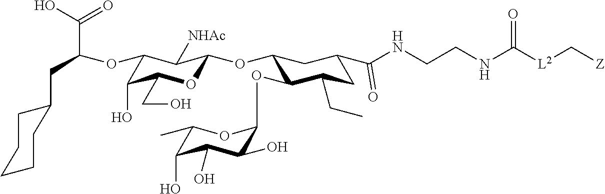

and pharmaceutically acceptable salts thereof.

In some embodiments, the compound of Formula (I) is chosen from:

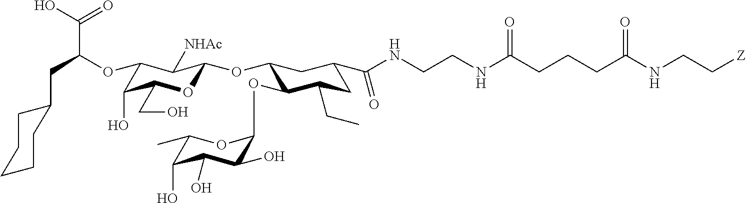

and pharmaceutically acceptable salts thereof.

In some embodiments, the compound of Formula (I) is chosen from:

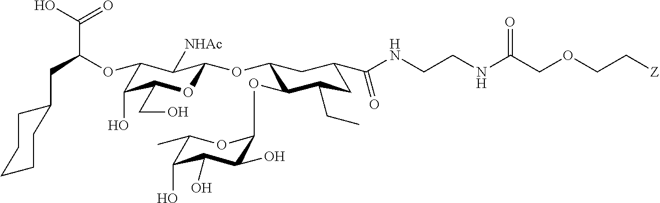

and pharmaceutically acceptable salts thereof.

In some embodiments, the compound of Formula (I) is chosen from:

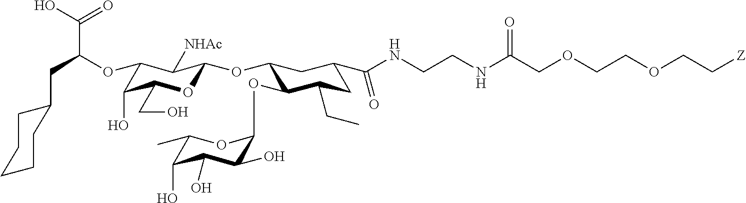

and pharmaceutically acceptable salts thereof.

In some embodiments, the compound of Formula (I) is chosen from: and pharmaceutically acceptable salts thereof.

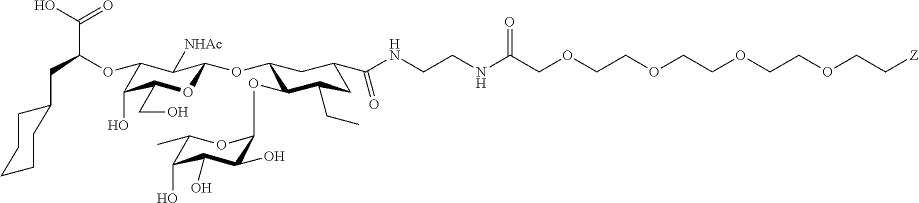

In some embodiments, the compound of Formula (I) is chosen from:

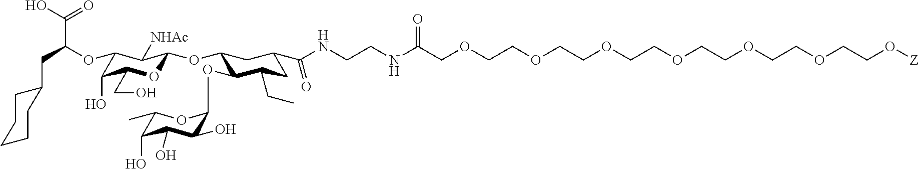

and pharmaceutically acceptable salts thereof.

In some embodiments, the compound of Formula (I) is chosen from:

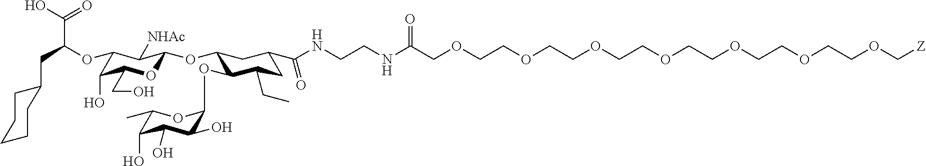

and pharmaceutically acceptable salts thereof.

In some embodiments, the compound of Formula (I) is chosen from:

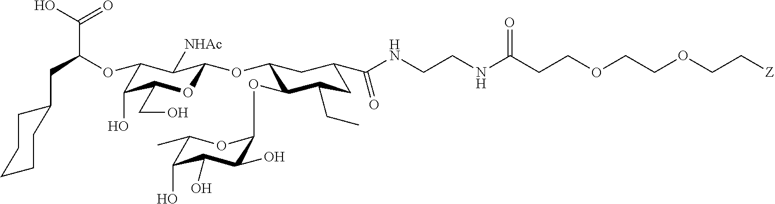

and pharmaceutically acceptable salts thereof.

In some embodiments, the compound of Formula (I) is chosen from:

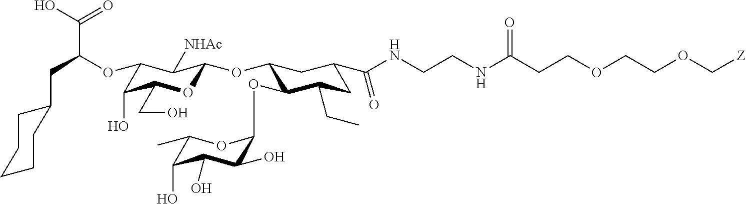

and pharmaceutically acceptable salts thereof.

In some embodiments, the compound of Formula (I) is chosen from:

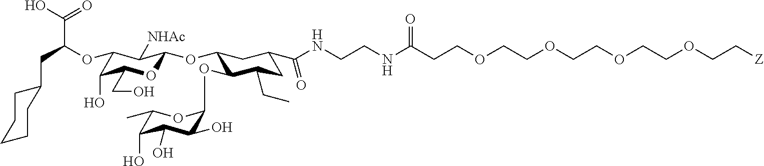

and pharmaceutically acceptable salts thereof.

In some embodiments, the compound of Formula (I) is chosen from:

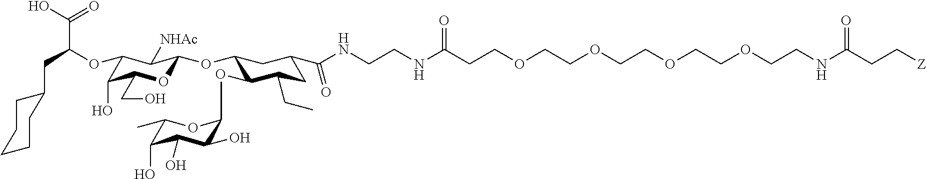

and pharmaceutically acceptable salts thereof.

In some embodiments, the compound of Formula (I) is chosen from:

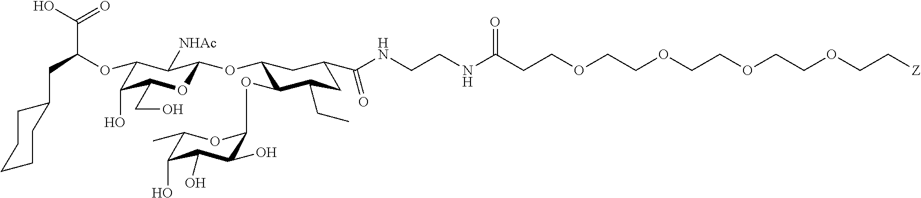

and pharmaceutically acceptable salts thereof.

In some embodiments, the compound of Formula (I) is chosen from:

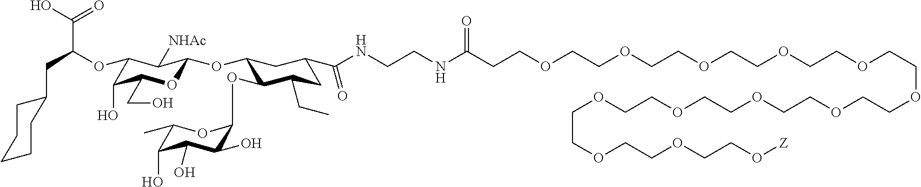

and pharmaceutically acceptable salts thereof.

In some embodiments, the compound of Formula (I) is chosen from:

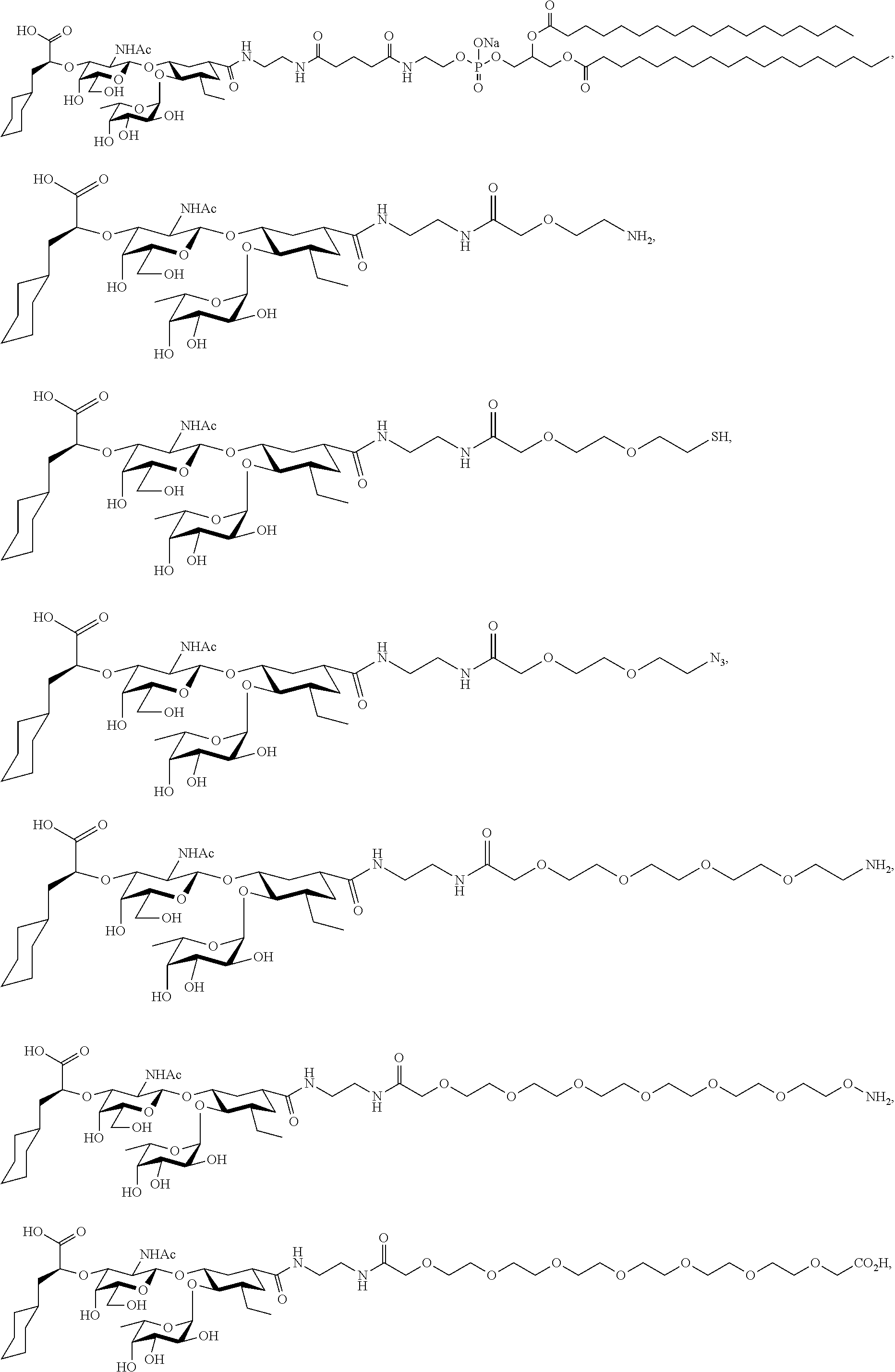

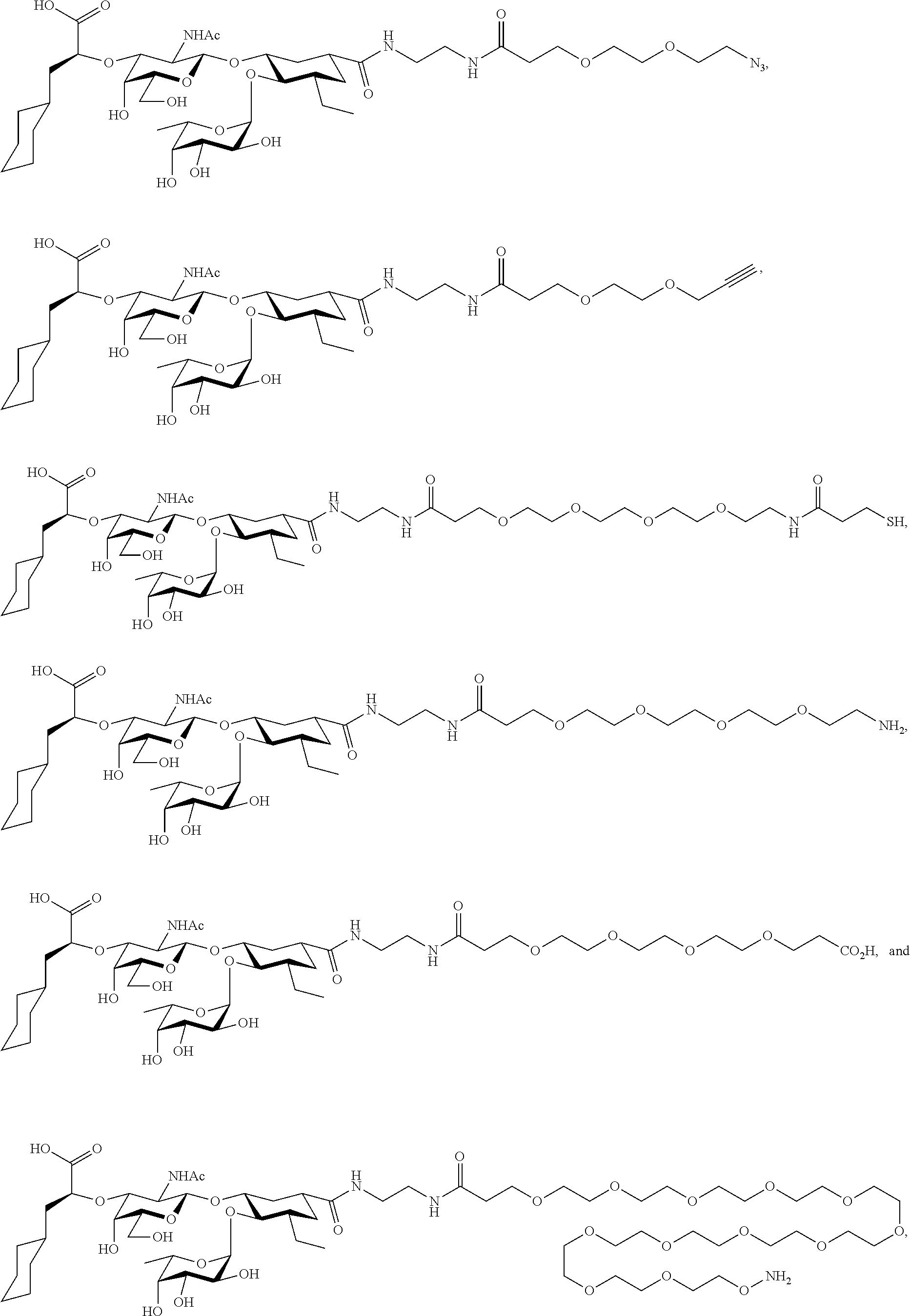

and pharmaceutically acceptable salts of any of the foregoing.

In some embodiments, at least one hydroxy group in the at least one compound of Formula (I) is protected with a hydroxy protecting group. In some embodiments, at least one carboxy group in the at least one compound of Formula (I) is protected with a carboxy protecting group. In some embodiments, at least one hydroxy group in the at least one compound of Formula (I) is protected with a hydroxy protecting group and at least one carboxy group in the at least one compound of Formula (I) is protected with a carboxy protecting group.

In some embodiments, the hydroxy protecting groups are independently chosen from formyl, acetyl, chloroacetyl, o-nitrophenylacetyl, o-nitrophenoxy-acetyl, trifluoroacetyl, acetoacetyl, 4-chlorobutyryl, isobutyryl, o-nitrocinnamoyl, picolinoyl, acylisothiocyanate, aminocaproyl, benzoyl, benzyl, β-methoxyethoxymethyl, dimethoxytrityl, methoxymethyl, methoxytrityl [(4-methoxyphenyl)diphenylmethyl], p-methoxybenzyl, methylthiomethyl, pivaloyl, tetrahydropyranyl, tetrahydrofuranyl, trityl, and silyl groups. In some embodiments, the carboxy protecting groups are chosen from methyl, benzyl, tert-butyl, and silyl groups.

In some embodiments, a process for making E-selectin ligand-bearing carriers of Formula (II) is provided, wherein the process comprises reacting or associating a compound of Formula (I), or protected form thereof, with a compound of Formula (III):

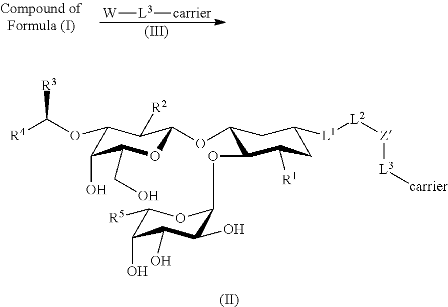

wherein
R¹, R², R³, R⁴, R⁵, L¹, and L² are as defined above;
L³ is chosen from a bond and linker groups;
W is chosen from lipids, nucleophiles, electrophiles, and groups capable of undergoing a cycloaddition reaction; and
Z′ is a moiety generated by the reaction or association of the Z group of the compound of Formula (I), or a protected form thereof, with the W group of the compound of Formula (III).

In some embodiments, L³ is a bond. In some embodiments, L³ is chosen from linker groups. In some embodiments, L³ is a linker group chosen from

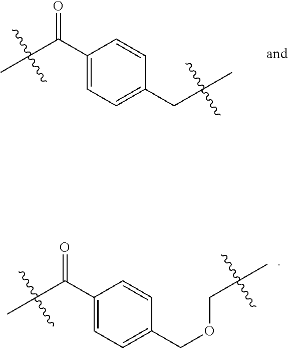 [SMILES: *Cc1ccc(C(*)=O)cc1 ; *COCc1ccc(C(*)=O)cc1]

In some embodiments, W is chosen from lipids. In some embodiments, W is chosen from glycerol-based lipids. In some embodiments, W is chosen from phospholipids. In some embodiments, W is chosen from symmetric phospholipids. In some embodiments, W is chosen from asymmetric phospholipids. In some embodiments, W is chosen from sphingolipids. In some embodiments, W is chosen from ceramides.

In some embodiments, W is chosen from phospholipids that have 16 to 30 carbon atoms. In some embodiments, W is chosen from phospholipids that have 16, 20, 22, 24, 26, 28, or 30 carbon atoms. In some embodiments, W is chosen from phospholipids that have 16 carbon atoms. In some embodiments, W is chosen from phospholipids that have 18 carbon atoms. In some embodiments, W is chosen from phospholipids that have 20 carbon atoms. In some embodiments, W is chosen from phospholipids that have 22 carbon atoms. In some embodiments, W is chosen from phospholipids that have 24 carbon atoms. In some embodiments, W is chosen from phospholipids that have 26 carbon atoms. In some embodiments, W is chosen from phospholipids that have 28 carbon atoms. In some embodiments, W is chosen from phospholipids that have 30 carbon atoms.

In some embodiments, W is chosen from saturated phospholipids. In some embodiments, W is chosen from unsaturated phospholipids. In some embodiments, W is chosen from phosphatidylcholine, phosphatidyethanolamine, phosphatidic acid, and phosphatidyl inositol. In some embodiments, W is chosen from phospholipid derivatives of 6-cis-octadecenoic, 9-cis-octadecenoic, 9-trans-octadecenoic, 9-cis-12-octadecadienoic, 9-cis-12-cis-15-cis-octadecatrienoic, 11-cis-eicosenoic, 5, 8, 11, 14 (all-cis) eicosatetraenoic, 13-cis-docosenoic, 4, 7, 10, 13, 16, 19 (all-cis) docosahexaenoic, and/or 15-cis-tetracosenoic acids. In some embodiments, W is chosen from phospholipid derivatives of stearoyl, oleoyl, linoleoyl, arachidonoyl, and/or docosahexaenoyl acids.

In some embodiments, W is chosen from nucleophiles. In some embodiments, the nucleophiles are chosen from amines, mercaptans, and alcohols. In some embodiments, W is chosen from amine groups.

In some embodiments, W is chosen from electrophiles. In some embodiments, the electrophiles are chosen from epoxides, aziridines, episulfides, sulfates, sulfonates, carbonates, lactones, lactams, halides, acid halides, and esters. In some embodiments, the electrophiles are chosen from epoxides. In some embodiments, the electrophiles are chosen from aziridines. In some embodiments, the electrophiles are chosen from episulfides. In some embodiments, the electrophiles are chosen from sulfates. In some embodiments, the electrophiles are chosen from sulfonates. In some embodiments, the electrophiles are chosen from carbonates. In some embodiments, the electrophiles are chosen from lactones. In some embodiments, the electrophiles are chosen from lactams. In some embodiments, the electrophiles are chosen from halides. In some embodiments, the electrophiles are chosen from acid halides. In some embodiments, the electrophiles are chosen from esters. In some embodiments, the esters are chosen from activated esters.

In some embodiments, W is chosen from esters. In some embodiments, the esters are chosen from anhydrides, carboxylic acid-succinimides, carboxylic acid phosphoesters, and carboxylic acid imidazolides. In some embodiments, W is chosen from esters formed with t-butanol, p-nitrophenol, 2,4-dinitrophenol, trichlorophenol, 1-hydroxy-1H-benzotriazole, 1-hydroxy-6-chloro-1H-benzotriazole, and N-hydroxysuccinimide.

In some embodiments, W is chosen from —C(═O)Y¹⁰ groups, wherein Y¹⁰ is chosen from H, —OH, —O^{t}Bu, C₂₋₈ alkenyl, C₁₋₈ haloalkyl, C₁₋₈ alkoxy, halo, C₆₋₁₈ aryloxy, C₁₋₁₃ heteroaryloxy, and C₂₋₁₂ heterocyclyloxy groups and wherein the C₁₋₈ alkoxy, C₆₋₁₈ aryloxy, C₁₋₁₃ heteroaryloxy, and C₂₋₁₂ heterocyclyloxy groups are optionally substituted with at least one halo group.

In some embodiments, W is chosen from groups capable of undergoing a cycloaddition reaction. In some embodiments, the groups capable of undergoing a cycloaddition reaction are chosen from alkenes, alkynes, dienes, dienophiles, 1,3-dipoles, and 1,3-dipolarophiles.

In some embodiments, the dienophiles are chosen from —C(═O)Y¹¹ groups, wherein Y¹¹ is chosen from alkene and alkyne groups, wherein said alkene and alkyne groups are optionally substituted with C₁₋₈ alkoxy, —C(═O)H, —C(═O)OY¹², and —C(═O)NY¹²Y¹³ groups, wherein Y¹² and Y¹³, which may be identical or different, are independently chosen from H and C₁₋₈ alkyl groups, or Y¹² and Y¹³ join together along with the nitrogen to which they are attached to form an optionally substituted, saturated or unsaturated, 3-10 membered ring.

In some embodiments, the 1,3-dipoles are chosen from nitrile ylides, nitrile imines, nitrile oxides, diazolalkanes, azides, azomethine ylides, azomethine imines, nitrones, carbonyl ylides, carbonyl imines, and carbonyl oxides.

In some embodiments, W is chosen from alkene and alkyne groups. In some embodiments, W is chosen from alkene groups. In some embodiments, W is chosen from alkyne groups. In some embodiments, W is —CH═CH₂. In some embodiments, W is —C≡CH.

In some embodiments, W is chosen from dienes and dienophiles. In some embodiments, W is chosen from dienes. In some embodiments, W is chosen from dienophiles.

In some embodiments, W is chosen from 1,3-dipoles and 1,3-dipolarophiles. In some embodiments, W is chosen from 1,3-dipoles. In some embodiments, W is chosen from 1,3-dipolarophiles.

In some embodiments, W is chosen from —N₃, —NH₂, —SH, —OH, —Cl, —Br, —I, —CH═CH₂, —C≡CH,

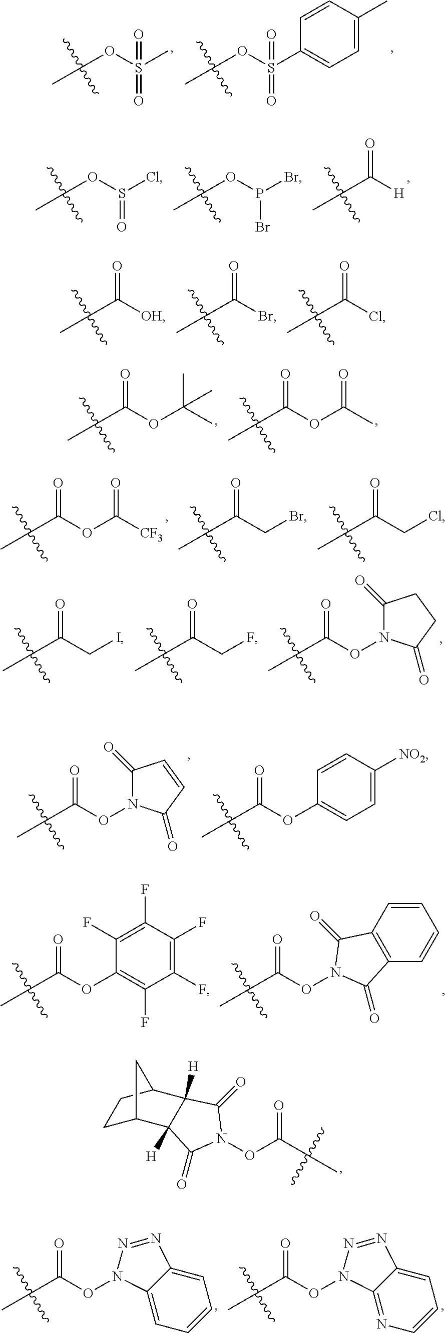

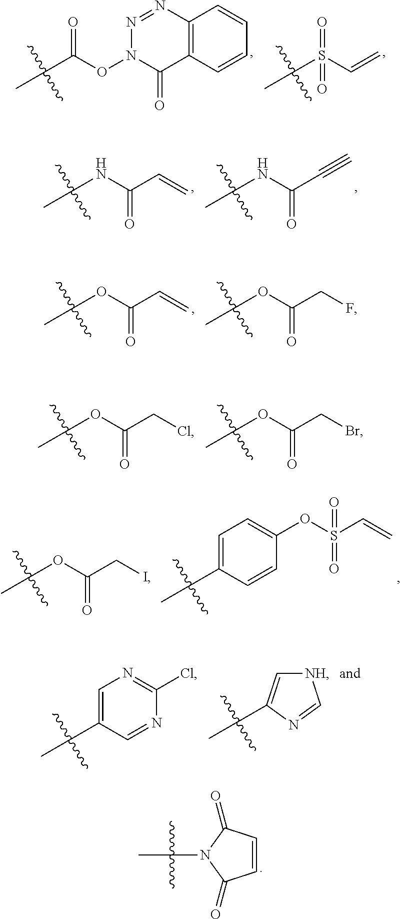

In some embodiments, W is —N(OH)H.

In some embodiments, W is —O—NH₂.

In some embodiments, W is —N₃.

In some embodiments, W is chosen from —NH₂, —SH, and —OH. In some embodiments, W is —NH₂. In some embodiments, W is —SH. In some embodiments, W is —OH.

In some embodiments, W is —C(═O)H. In some embodiments, W is —C(═O)OH. In some embodiments, W is —C(═O)C₁. In some embodiments, W is —C(═O)O^{t}Bu.

In some embodiments, Z′ is chosen from a lipid-lipid non-covalent association, —C(═O)HN—, —CH₂NH—, —CH₂S—,

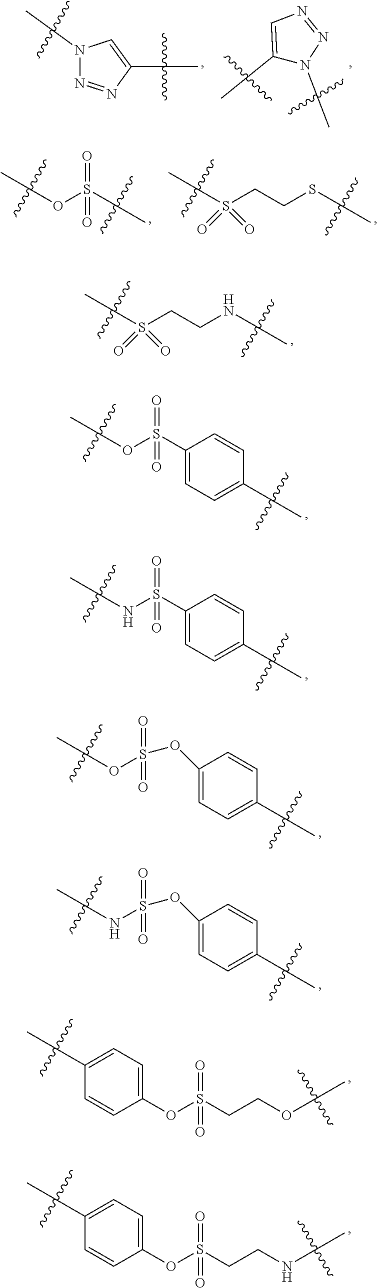

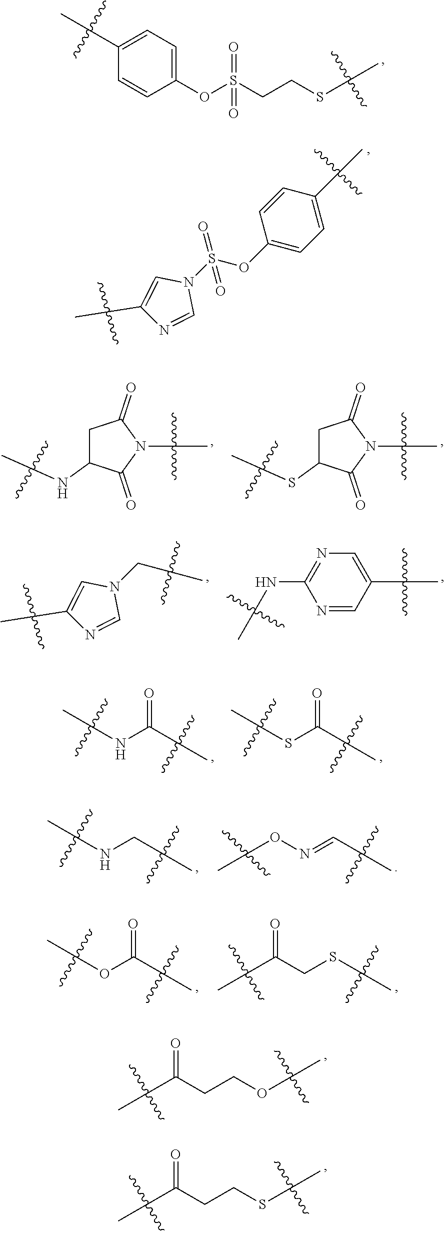

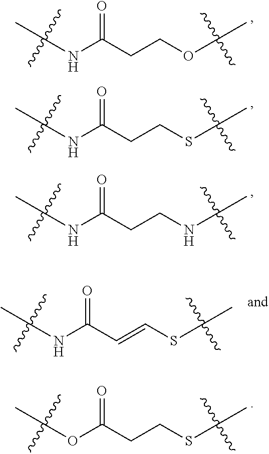

In some embodiments, Z′ is

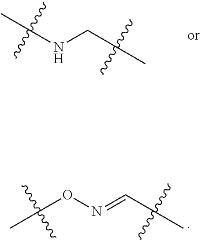

In some embodiments, a process for making E-selectin ligand-bearing carriers of Formula (II) is provided, wherein the process comprises reacting or associating a compound of Formula (I), or a protected form thereof, with a compound of Formula (III):

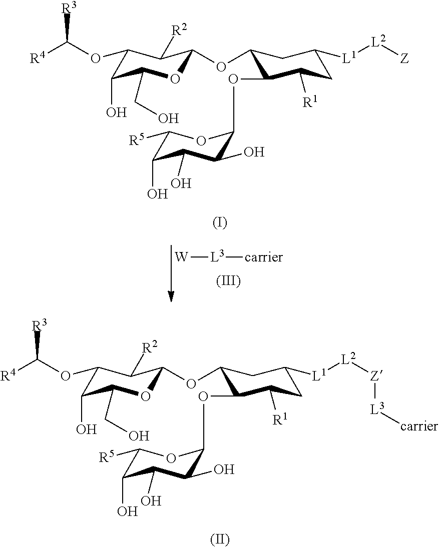

wherein
R¹, R², R³, R⁴, R⁵, R⁶, L¹, L², and L³ are as defined above; and
Z, W, and Z′ are as shown below:

| Z | W | Z′ |
|---|---|---|
| lipid | lipid | lipid-lipid non-covalent |
| | | association |
| —N₃ | HC≡C— | 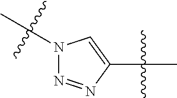 [SMILES: *c1cn(*)nn1] |
| | HC≡C— | 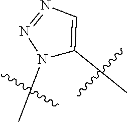 [SMILES: *c1cnnn1*] |
| —C≡CH | N₃— | 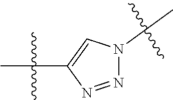 [SMILES: *c1cn(*)nn1] |
| —C≡CH | | 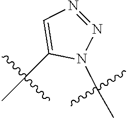 [SMILES: *c1cnnn1*] |
| —NH₂ | HO(O═)C— | —NH(O═)C— |
| | Cl(O═)C— | |
| | 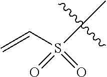 [SMILES: *S(=O)(=O)C=C] | 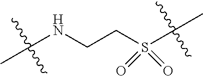 [SMILES: *NCCS(*)(=O)=O] |
| | 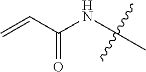 [SMILES: *NC(=O)C=C] | 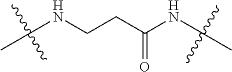 [SMILES: *NCCC(=O)N*] |
| | 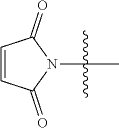 [SMILES: *N1C(=O)C=CC1=O] | 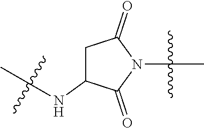 [SMILES: *NC1CC(=O)N(*)C1=O] |
| | BrH₂C— | —NHCH₂— |
| | 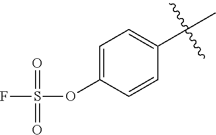 [SMILES: *c1ccc(OS(=O)(=O)F)cc1] | 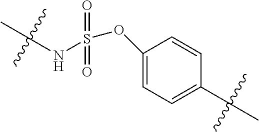 [SMILES: *NS(=O)(=O)Oc1ccc(*)cc1] |
| | 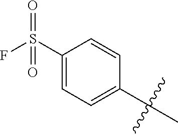 [SMILES: *c1ccc(S(=O)(=O)F)cc1] | 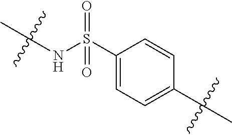 [SMILES: *NS(=O)(=O)c1ccc(*)cc1] |
| | 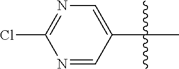 [SMILES: *c1cnc(Cl)nc1] | 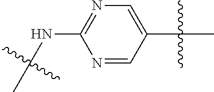 [SMILES: *Nc1ncc(*)cn1] |
| | 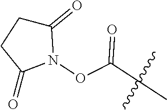 [SMILES: *C(=O)ON1C(=O)CCC1=O] | 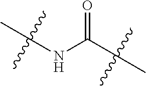 [SMILES: *NC(*)=O] |
| | 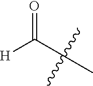 [SMILES: *C=O] | 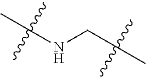 [SMILES: *CN*] |
| —O—NH₂ | 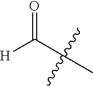 [SMILES: *C=O] | 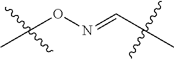 [SMILES: */C=N/O*] |
| —SH | 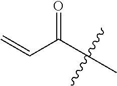 [SMILES: *C(=O)C=C] | 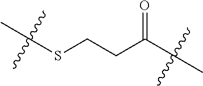 [SMILES: *SCCC(*)=O] |
| | 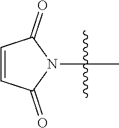 [SMILES: *N1C(=O)C=CC1=O] | 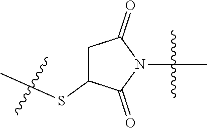 [SMILES: *SC1CC(=O)N(*)C1=O] |
| | 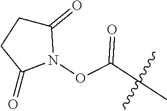 [SMILES: *C(=O)ON1C(=O)CCC1=O] | 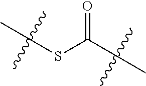 [SMILES: *SC(*)=O] |
| | 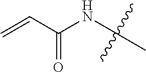 [SMILES: *NC(=O)C=C] | 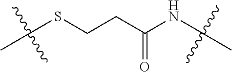 [SMILES: *NC(=O)CCS*] |
| | 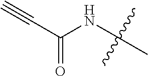 [SMILES: *NC(=O)C#C] | 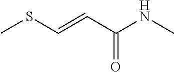 |
| | 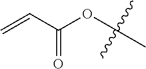 [SMILES: *OC(=O)C=C] | 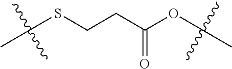 [SMILES: *OC(=O)CCS*] |
| | 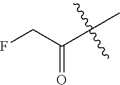 [SMILES: *C(=O)CF] | 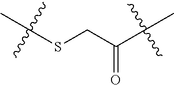 [SMILES: *SCC(*)=O] |
| | 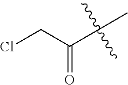 [SMILES: *C(=O)CCl] | |
| | 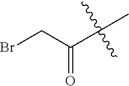 [SMILES: *C(=O)CBr] | |
| | 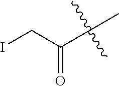 [SMILES: *C(=O)CI] | |
| | 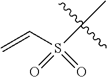 [SMILES: *S(=O)(=O)C=C] | 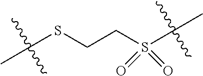 [SMILES: *SCCS(*)(=O)=O] |
| | BrH₂C— | —SH₂C— |
| | 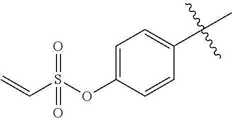 [SMILES: *c1ccc(OS(=O)(=O)C=C)cc1] | 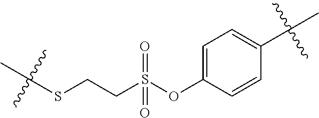 [SMILES: *SCCS(=O)(=O)Oc1ccc(*)cc1] |
| —OH | 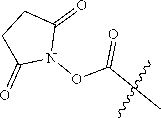 [SMILES: *C(=O)ON1C(=O)CCC1=O] | 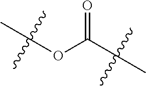 [SMILES: *OC(*)=O] |
| | 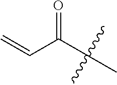 [SMILES: *C(=O)C=C] | 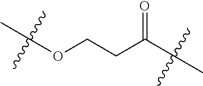 [SMILES: *OCCC(*)=O] |
| | 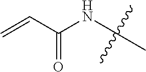 [SMILES: *NC(=O)C=C] | 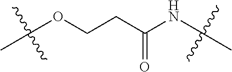 [SMILES: *NC(=O)CCO*] |
| | 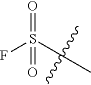 [SMILES: *S(=O)(=O)F] | 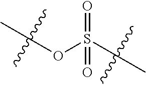 [SMILES: *OS(*)(=O)=O] |
| | 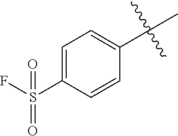 [SMILES: *c1ccc(S(=O)(=O)F)cc1] | 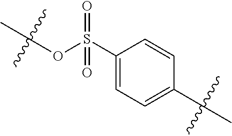 [SMILES: *OS(=O)(=O)c1ccc(*)cc1] |
| | 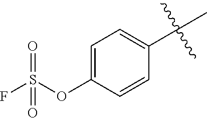 [SMILES: *c1ccc(OS(=O)(=O)F)cc1] | 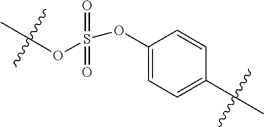 [SMILES: *OS(=O)(=O)Oc1ccc(*)cc1] |
| 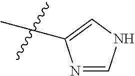 [SMILES: *c1c[nH]cn1] | BrH₂C— | 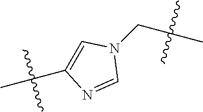 [SMILES: *Cn1cnc(*)c1] |
| | 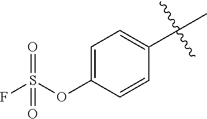 [SMILES: *c1ccc(OS(=O)(=O)F)cc1] | 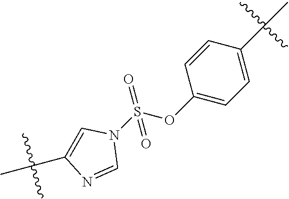 [SMILES: *c1ccc(OS(=O)(=O)n2cnc(*)c2)cc1] |
| —C(═O)OH | H₂N— | —C(═O)HN— |
| —C(═O)Cl | | —C(═O)HN— |
| 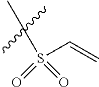 [SMILES: *S(=O)(=O)C=C] | H₂N— | 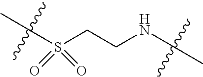 [SMILES: *NCCS(*)(=O)=O] |
| | HS— | 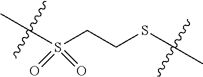 [SMILES: *SCCS(*)(=O)=O] |
| 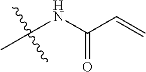 [SMILES: *NC(=O)C=C] | H₂N— | 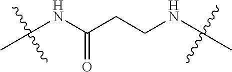 [SMILES: *NCCC(=O)N*] |
| | HS— | 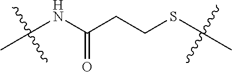 [SMILES: *NC(=O)CCS*] |
| | HO— | 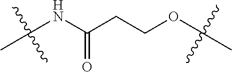 [SMILES: *NC(=O)CCO*] |
| 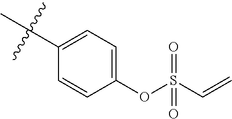 [SMILES: *c1ccc(OS(=O)(=O)C=C)cc1] | H₂N— | 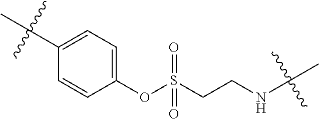 [SMILES: *NCCS(=O)(=O)Oc1ccc(*)cc1] |
| | HS— | 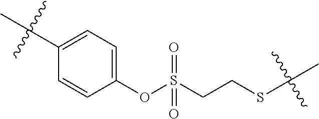 [SMILES: *SCCS(=O)(=O)Oc1ccc(*)cc1] |
| | HO— | 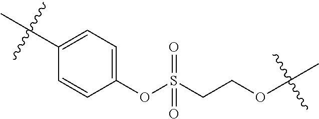 [SMILES: *OCCS(=O)(=O)Oc1ccc(*)cc1] |
| 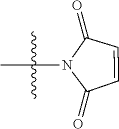 [SMILES: *N1C(=O)C=CC1=O] | H₂N— | 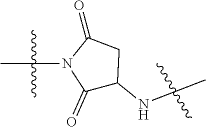 [SMILES: *NC1CC(=O)N(*)C1=O] |
| | HS— | 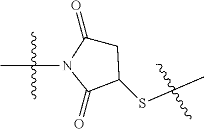 [SMILES: *SC1CC(=O)N(*)C1=O] |
| —CH₂Br | H₂N— | —CH₂NH— |
| | HS— | —CH₂S— |
| | 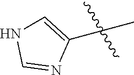 [SMILES: *c1c[nH]cn1] | 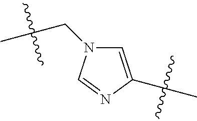 [SMILES: *Cn1cnc(*)c1] |
| 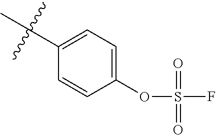 [SMILES: *c1ccc(OS(=O)(=O)F)cc1] | H₂N— | 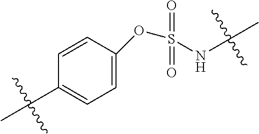 [SMILES: *NS(=O)(=O)Oc1ccc(*)cc1] |
| | HO— | 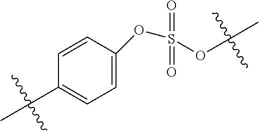 [SMILES: *OS(=O)(=O)Oc1ccc(*)cc1] |
| | 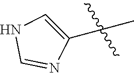 [SMILES: *c1c[nH]cn1] | 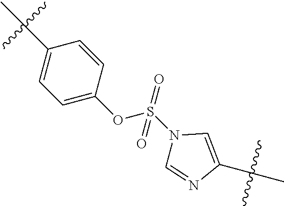 [SMILES: *c1ccc(OS(=O)(=O)n2cnc(*)c2)cc1] |
| 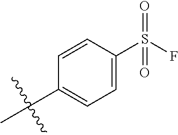 [SMILES: *c1ccc(S(=O)(=O)F)cc1] | H₂N— | 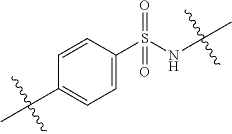 [SMILES: *NS(=O)(=O)c1ccc(*)cc1] |
| | HO— | 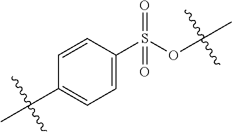 [SMILES: *OS(=O)(=O)c1ccc(*)cc1] |
| 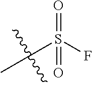 [SMILES: *S(=O)(=O)F] | HO— | 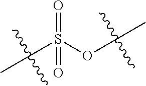 [SMILES: *OS(*)(=O)=O] |
| 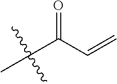 [SMILES: *C(=O)C=C] | HO— | 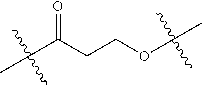 [SMILES: *OCCC(*)=O] |
| | HS— | 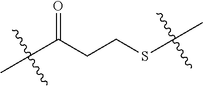 [SMILES: *SCCC(*)=O] |
| 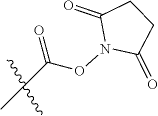 [SMILES: *C(=O)ON1C(=O)CCC1=O] | H₂N— | 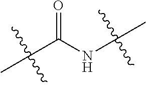 [SMILES: *NC(*)=O] |
| | HS— | 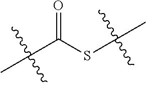 [SMILES: *SC(*)=O] |
| | HO— | 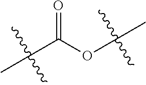 [SMILES: *OC(*)=O] |
| 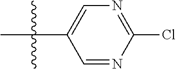 [SMILES: *c1cnc(Cl)nc1] | H₂N— | 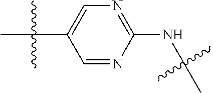 [SMILES: *Nc1ncc(*)cn1] |
| 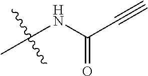 [SMILES: *NC(=O)C#C] | HS— | 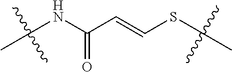 [SMILES: *NC(=O)/C=C/S*] |
| 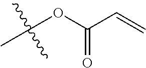 [SMILES: *OC(=O)C=C] | | 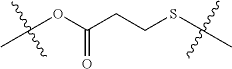 [SMILES: *OC(=O)CCS*] |
| 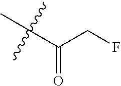 [SMILES: *C(=O)CF] | | 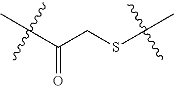 [SMILES: *SCC(*)=O] |
| 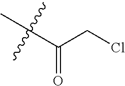 [SMILES: *C(=O)CCl] | | 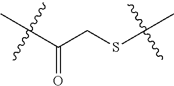 [SMILES: *SCC(*)=O] |
| 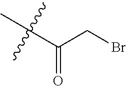 [SMILES: *C(=O)CBr] | | 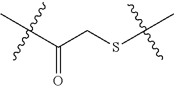 [SMILES: *SCC(*)=O] |
| 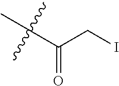 [SMILES: *C(=O)CI] | | 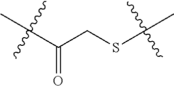 [SMILES: *SCC(*)=O] |
| 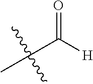 [SMILES: *C=O] | H₂N— | 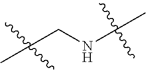 [SMILES: *CN*] |
| | H₂N—O— | 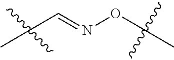 [SMILES: */C=N/O*] |

In some embodiments, the carriers are chosen from particles, nanoparticles, liposomes, beads, proteins, polysaccharides, lipids, metal chelating agents, and combinations thereof. In some embodiments, the carriers are chosen from lipid-based, protein-based, nucleic acid based, and carbohydrate-based carriers. In some embodiments, the carriers are chosen from carriers composed of polymer and/or non-polymer molecules. In some embodiments, the carriers are chosen from macromolecular carriers. In some embodiments, the carriers comprise crosslinking chains of molecules. In some embodiments, the crosslinking chains of molecules are chosen from nucleic acids. In some embodiments, the carriers are chosen from polyamino-based carriers.

In some embodiments, the carriers are chosen from lipid-based nanoparticles, polymeric nanoparticles, metallic nanoparticles, surfactant-based emulsion, dendrimers, and nanoparticles.

In some embodiments, the carriers are formed by self-assembly. In some embodiments, self-assembly occurs in vitro. In some embodiments, self-assembly occurs in vivo. In some embodiments, the carriers are formed using amphiphilic biomaterials which orient themselves with respect to one another to form carriers of predictable dimension, constituents, and placement of constituents.

In some embodiments, the carriers are chosen from carriers having a mean geometric diameter that is less than 500 nm. In some embodiments, the carriers are chosen from carriers having a mean geometric diameter that is greater than 50 nm but less than 500 nm.

In some embodiments, the carriers are nanoparticles.

In embodiments, the Z group of Formula (I) is a lipid and the carrier is chosen from liposomes.

In some embodiments, the carrier is a metal chelating agent.

In some embodiments, the metal chelating agent is

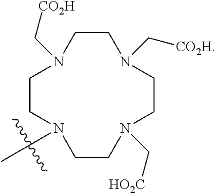 [SMILES: *N1CCN(CC(=O)O)CCN(CC(=O)O)CCN(CC(=O)O)CC1]

In some embodiments, the metal chelating agent is

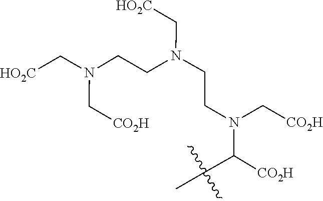 [SMILES: *C(C(=O)O)N(CCN(CCN(CC(=O)O)CC(=O)O)CC(=O)O)CC(=O)O]

In some embodiments, a method for treatment and/or prevention of at least one disease, disorder, or condition is disclosed, the method comprising administering to a subject in need thereof an effective amount of at least one compound of Formula (II) or a composition comprising the same, wherein at least one therapeutic agent is directly or indirectly linked to or associated with said at least one compound of Formula (II).

In some embodiments, a method for diagnosing at least one disease, disorder, or condition is disclosed, the method comprising administering to a subject in need thereof an effective amount of at least one compound of Formula (II) or a composition comprising the same, wherein at least one diagnostic agent is directly or indirectly linked to or associated with said at least one compound of Formula (II).

In some embodiments, a method for treatment and/or prevention of at least one disease, disorder, or condition is disclosed, the method comprising administering to a subject in need thereof an effective amount of at least one compound of Formula (II) or a composition comprising the same, wherein at least one radiopharmaceutical is directly or indirectly linked to or associated with said at least one compound of Formula (II).

In some embodiments, the at least one radiopharmaceutical is ¹⁷⁷Lu. In some embodiments, the at least one radiopharmaceutical is ¹¹¹In.

In some embodiments, the at least one disease, disorder, or condition is chosen from cancers, inflammatory diseases, metastasis, and angiogenesis. In some embodiments, the cancer is pancreatic cancer. In some embodiments, the cancer is prostate cancer. In some embodiments, the cancer is breast cancer. In some embodiments, the cancer is lung cancer. In some embodiments, the cancer is chosen from melanomas. In some embodiments, the cancer is chosen from hematological malignancies. In some embodiments, the cancer is leukemia.

In some embodiments, a method for diagnosing or imaging E-selectin expressing tissues is disclosed, the method comprising administering to a subject in need thereof an effective amount of at least one compound of Formula (II) or a composition comprising the same, wherein at least one diagnostic agent or at least one imaging agent is directly or indirectly linked to or associated with said at least one compound of Formula (II).

In some embodiments, the at least one imaging agent is Gd³⁺. In some embodiments, the at least one imaging agent is technetium 99 m. In some embodiments, the at least one imaging agent is chosen from cross-linked iron oxide superparamagnetic nanoparticles. In some embodiments, the at least one imaging agent is capable of imaging blood vessels. In some embodiments, the at least one imaging agent is capable of imaging angiogenesis.

In some embodiments, the method for diagnosing or imaging E-selectin expressing tissues comprises imaging angiogenesis in the eye of age-related macular degeneration patients. In some embodiments, the method for diagnosing or imaging E-selectin expressing tissues comprises imaging angiogenesis in a tumor.

Whenever a term in the specification is identified as a range (e.g., C₁₋₄ alkyl) or “ranging from”, the range independently discloses and includes each element of the range. As a non-limiting example, C₁₋₄ alkyl groups includes, independently, C₁ alkyl groups, C₂ alkyl groups, C₃ alkyl groups, and C₄ alkyl groups. As another non-limiting example, “n is chosen from integers ranging from 0 to 2” includes, independently, 0, 1, and 2.

The term “at least one” refers to one or more, such as one, two, etc. For example, the term “at least one C₁₋₄ alkyl group” refers to one or more C₁₋₄ alkyl groups, such as one C₁₋₄ alkyl group, two C₁₋₄ alkyl groups, etc.

The term “1,3-dipole” includes compounds that contains a consecutive series of three atoms, a-b-c, where atom a contains a sextet of electrons in its outer shell and atom c contains an octet with at least one unshared pair of electrons in its outer shell. Because molecules that have six electrons in the outer shell of an atom are typically unstable, the a-b-c atom example is one canonical structure of a resonance hybrid, where at least one structure can be drawn. The term 1,3-dipoles include those in which one of the canonical forms has a double bond on the sextet atom (atom a) and the other canonical form has a triple bond on that atom:

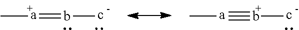

The term 1,3-dipoles also includes those in which the dipolar canonical form has a single bond on the sextet atom (atom a) and the other canonical form has a double bond on that atom:

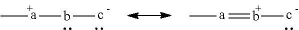

The term “1,3-dipolarophile” includes dienophiles and dienes.

The term “activated ester” means an ester derivative that has sufficient reactivity such that it can react with a nucleophile (e.g., an amino group).

The term “alkyl” includes saturated straight, branched, and cyclic (also identified as cycloalkyl), primary, secondary, and tertiary hydrocarbon groups. Non-limiting examples of alkyl groups include methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless stated otherwise specifically in the specification, an alkyl group may be optionally substituted.

The term “alkenyl” includes straight, branched, and cyclic hydrocarbon groups comprising at least one double bond. The double bond of an alkenyl group can be unconjugated or conjugated with another unsaturated group. Non-limiting examples of alkenyl groups include vinyl, allyl, butenyl, pentenyl, hexenyl, butadienyl, pentadienyl, hexadienyl, 2-ethylhexenyl, and cyclopent-1-en-1-yl. Unless stated otherwise specifically in the specification, an alkenyl group may be optionally substituted.

The term “alkynyl” includes straight and branched hydrocarbon groups comprising at least one triple bond. The triple bond of an alkynyl group can be unconjugated or conjugated with another unsaturated group. Non-limiting examples of alkynyl groups include ethynyl, propynyl, butynyl, pentynyl, and hexynyl. Unless stated otherwise specifically in the specification, an alkynyl group may be optionally substituted.

The term “aryl” includes hydrocarbon ring system groups comprising at least 6 carbon atoms and at least one aromatic ring. The aryl group may be a monocyclic, bicyclic, tricyclic, or tetracyclic ring system, which may include fused or bridged ring systems. Non-limiting examples of aryl groups include aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

The term “carboxy protecting group” includes groups known in the art to protect an acidic hydrogen of a carboxyl group against undesirable reaction during synthetic procedures, e.g., to block or protect the acid functionality while the reactions involving other functional sites of the compound are carried out, and to be removable.

The term “diene” includes a molecule bearing two conjugated double bonds. The diene may also be non-conjugated, if the geometry of the molecule is constrained so as to facilitate a cycloaddition reaction (see Cookson J. Chem. Soc. 5416 (1964), which is incorporated by reference in its entirety). The atoms forming these double bonds can be carbon or a heteroatom or any combination thereof.

The term “dienophile” includes a molecule bearing an alkene group, or a double bond between a carbon and a heteroatom, or a double bond between two heteroatoms.

The term “electrophile” includes chemical moieties which can accept a pair of electrons from a nucleophile. Electrophiles include cyclic compounds such as epoxides, aziridines, episulfides, cyclic sulfates, carbonates, lactones, lactams and the like. Non-cyclic electrophiles include sulfates, sulfonates (e.g. tosylates), chlorides, bromides, iodides, and the like.

The term “halo” or “halogen” includes fluoro, chloro, bromo, and iodo.

The term “haloalkyl” includes alkyl groups, as defined herein, substituted by at least one halogen, as defined herein. Non-limiting examples of haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl. A “fluoroalkyl” is a haloalkyl wherein at least one halogen is fluoro. Unless stated otherwise specifically in the specification, a haloalkyl group may be optionally substituted.

The term “haloalkenyl” includes alkenyl groups, as defined herein, substituted by at least one halogen, as defined herein. Non-limiting examples of haloalkenyl groups include fluoroethenyl, 1,2-difluoroethenyl, 3-bromo-2-fluoropropenyl, and 1,2-dibromoethenyl. A “fluoroalkenyl” is a haloalkenyl substituted with at least one fluoro group. Unless stated otherwise specifically in the specification, a haloalkenyl group may be optionally substituted.

The term “haloalkynyl” includes alkynyl groups, as defined herein, substituted by at least one halogen, as defined herein. Non-limiting examples include fluoroethynyl, 1,2-difluoroethynyl, 3-bromo-2-fluoropropynyl, and 1,2-dibromoethynyl. A “fluoroalkynyl” is a haloalkynyl wherein at least one halogen is fluoro. Unless stated otherwise specifically in the specification, a haloalkynyl group may be optionally substituted.

The term “heterocyclyl” or “heterocyclic ring” includes 3- to 24-membered saturated or partially unsaturated non-aromatic ring groups comprising 2 to 23 ring carbon atoms and 1 to 8 ring heteroatom(s) each independently chosen from N, O, and S. Unless stated otherwise specifically in the specification, the heterocyclyl groups may be monocyclic, bicyclic, tricyclic or tetracyclic ring systems, which may include fused or bridged ring systems, and may be partially or fully saturated; any nitrogen, carbon or sulfur atom(s) in the heterocyclyl group may be optionally oxidized; any nitrogen atom in the heterocyclyl group may be optionally quaternized. Non-limiting examples of heterocyclic ring include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

The term “heteroaryl” includes 5- to 14-membered ring groups comprising 1 to 13 ring carbon atoms and 1 to 6 ring heteroatom(s) each independently chosen from N, O, and S, and at least one aromatic ring. Unless stated otherwise specifically in the specification, the heteroaryl group may be a monocyclic, bicyclic, tricyclic or tetracyclic ring system, which may include fused or bridged ring systems; and the nitrogen, carbon, or sulfur atoms in the heteroaryl radical may be optionally oxidized; the nitrogen atom may be optionally quaternized. Non-limiting examples include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

The term “hydroxy protecting group” includes groups known in the art to protect a hydroxyl group against undesirable reaction during synthetic procedures and to be removable. The use of hydroxy protecting groups is well known in the art for protecting groups against undesirable reactions during a synthetic procedure and many such protecting groups are known.

The term “nucleophile” includes a chemical moiety having a reactive pair of electrons. Examples of nucleophiles include uncharged compounds such as amines, mercaptans and alcohols, and charged moieties such as alkoxides, thiolates, carbanions, and a variety of organic and inorganic anions. Illustrative anionic nucleophiles include simple anions such as azide, cyanide, thiocyanate, acetate, formate or chloroformate, and bisulfite. Organometallic reagents such as organocuprates, organozincs, organolithiums, Grignard reagents, enolates, acetylides, and the like may, under appropriate reaction conditions, be suitable nucleophiles.

The term “pharmaceutically acceptable salts” includes both acid and base addition salts. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Non-limiting examples of pharmaceutically acceptable base addition salts include sodium, potassium, lithium, ammonium (substituted and unsubstituted), calcium, magnesium, iron, zinc, copper, manganese, and aluminum salts. Pharmaceutically acceptable salts may, for example, be obtained using standard procedures well known in the field of pharmaceuticals.

The term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto, and amino functional groups in the compounds described herein.

“Self-assembly” refers to the process of the formation of, for example, a carrier using components that will orient themselves in a predictable manner forming carriers predictably and reproducibly.

The term “substituted” includes the situation where, in any of the above groups, at least one hydrogen atom is replaced by a non-hydrogen atom such as, for example, a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the above groups, at least one hydrogen atom is replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

This application contemplates all the isomers of the compounds disclosed herein. “Isomer” as used herein includes optical isomers (such as stereoisomers, e.g., enantiomers and diastereoisomers), geometric isomers (such as Z (zusammen) or E (entgegen) isomers), and tautomers. The present disclosure includes within its scope all the possible geometric isomers, e.g., Z and E isomers (cis and trans isomers), of the compounds as well as all the possible optical isomers, e.g., diastereomers and enantiomers, of the compounds. Furthermore, the present disclosure includes in its scope both the individual isomers and any mixtures thereof, e.g., racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof conventional resolution methods, e.g., fractional crystallization, may be used.

The present disclosure includes within its scope all possible tautomers. Furthermore, the present disclosure includes in its scope both the individual tautomers and any mixtures thereof. Each compound disclosed herein includes within its scope all possible tautomeric forms. Furthermore, each compound disclosed herein includes within its scope both the individual tautomeric forms and any mixtures thereof. With respect to the methods, uses and compositions of the present application, reference to a compound or compounds is intended to encompass that compound in each of its possible isomeric forms and mixtures thereof. Where a compound of the present application is depicted in one tautomeric form, that depicted structure is intended to encompass all other tautomeric forms.

Biological activity of the E-selectin ligands and/or E-selectin ligand-bearing carriers described herein may be determined, for example, by performing at least one in vitro and/or in vivo study routinely practiced in the art.

Conditions for a particular assay include temperature, buffers (including salts, cations, media), and other components that maintain the integrity of any cell used in the assay and the compound, which a person of ordinary skill in the art will be familiar and/or which can be readily determined. A person of ordinary skill in the art also readily appreciates that appropriate controls can be designed and included when performing the in vitro methods and in vivo methods described herein.

The source of a compound that is characterized by at least one assay and techniques described herein and in the art may be a biological sample that is obtained from a subject who has been treated with the compound. The cells that may be used in the assay may also be provided in a biological sample. A “biological sample” may include a sample from a subject, and may be a blood sample (from which serum or plasma may be prepared), a biopsy specimen, one or more body fluids (e.g., lung lavage, ascites, mucosal washings, synovial fluid, urine), bone marrow, lymph nodes, tissue explant, organ culture, or any other tissue or cell preparation from the subject or a biological source. A biological sample may further include a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. In some embodiments, the subject or biological source may be a human or non-human animal, a primary cell culture (e.g., immune cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiable cell lines, transformed cell lines, and the like.

As described herein, methods for characterizing E-selectin ligands and/or E-selectin ligand-bearing carriers include animal model studies.

As understood by a person of ordinary skill in the medical art, the terms “treat” and “treatment” include medical management of a disease, disorder, or condition of a subject (i.e., patient, individual) (see, e.g., Stedman's Medical Dictionary). In general, an appropriate dose and treatment regimen provide at least one of the compounds of the present disclosure in an amount sufficient to provide therapeutic and/or prophylactic benefit. For both therapeutic treatment and prophylactic or preventative measures, therapeutic and/or prophylactic benefit includes, for example, an improved clinical outcome, wherein the object is to prevent or slow or retard (lessen) an undesired physiological change or disorder, or to prevent or slow or retard (lessen) the expansion or severity of such disorder. As discussed herein, beneficial or desired clinical results from treating a subject include, but are not limited to, abatement, lessening, or alleviation of symptoms that result from or are associated with the disease, condition, or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can include prolonging survival when compared to expected survival if a subject was not receiving treatment. Subjects in need of treatment include those who already have the disease, condition, or disorder as well as subjects prone to have or at risk of developing the disease, condition, or disorder, and those in which the disease, condition, or disorder is to be prevented (i.e., decreasing the likelihood of occurrence of the disease, disorder, or condition).

In some embodiments of the methods described herein, the subject is a human. In some embodiments of the methods described herein, the subject is a non-human animal. A subject in need of treatment as described herein may exhibit at least one symptom or sequelae of the disease, disorder, or condition described herein or may be at risk of developing the disease, disorder, or condition. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

The effectiveness of the compounds of the present disclosure in treating and/or preventing a disease, disorder, or condition described herein can readily be determined by a person of ordinary skill in the medical and clinical arts. Determining and adjusting an appropriate dosing regimen (e.g., adjusting the amount of compound per dose and/or number of doses and frequency of dosing) can also readily be performed by a person of ordinary skill in the medical and clinical arts. One or any combination of diagnostic methods, including physical examination, assessment and monitoring of clinical symptoms, and performance of analytical tests and methods described herein, may be used for monitoring the health status of the subject.

Also provided herein are pharmaceutical compositions comprising at least one compound of Formula (I). In some embodiments, the pharmaceutical composition further comprises at least one additional pharmaceutically acceptable ingredient.

In pharmaceutical dosage forms, any one or more of the compounds of the present disclosure may be administered in the form of a pharmaceutically acceptable derivative, such as a salt, and/or it/they may also be used alone and/or in appropriate association, as well as in combination, with other pharmaceutically active compounds.

An effective amount or therapeutically effective amount refers to an amount of a compound of the present disclosure or a composition comprising at least one such compound that, when administered to a subject, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the subject.

An effective amount or therapeutically effective amount refers to an amount of a compound of the present disclosure or a composition comprising at least one such compound that, when administered to a subject, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the subject.

The minimum dose that is sufficient to provide effective therapy may be used in some embodiments. Subjects may generally be monitored for therapeutic effectiveness using assays suitable for the disease or condition being treated or prevented, which assays will be familiar to those having ordinary skill in the art and are described herein. The level of a compound that is administered to a subject may be monitored by determining the level of the compound (or a metabolite of the compound) in a biological fluid, for example, in the blood, blood fraction (e.g., serum), and/or in the urine, and/or other biological sample from the subject. Any method practiced in the art to detect the compound, or metabolite thereof, may be used to measure the level of the compound during the course of a therapeutic regimen.

The dose of a compound described herein may depend upon the subject's condition, that is, stage of the disease, severity of symptoms caused by the disease, general health status, as well as age, gender, and weight, and other factors apparent to a person of ordinary skill in the medical art. Similarly, the dose of the therapeutic for treating a disease or disorder may be determined according to parameters understood by a person of ordinary skill in the medical art.

Pharmaceutical compositions may be administered in any manner appropriate to the disease or disorder to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the pharmaceutical composition(s) as described herein in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail above).

The pharmaceutical compositions described herein may be administered to a subject in need thereof by any one of several routes that effectively delivers an effective amount of the compound. Non-limiting suitable administrative routes include topical, oral, nasal, intrathecal, enteral, buccal, sublingual, transdermal, rectal, vaginal, intraocular, subconjunctival, sublingual, and parenteral administration, including subcutaneous, intravenous, intramuscular, intrasternal, intracavernous, intrameatal, and intraurethral injection and/or infusion.

The pharmaceutical compositions described herein may be sterile aqueous or sterile non-aqueous solutions, suspensions or emulsions, and may additionally comprise at least one pharmaceutically acceptable excipient (i.e., a non-toxic material that does not interfere with the activity of the active ingredient). Such compositions may be in the form of a solid, liquid, or gas (aerosol). Alternatively, the compositions described herein may be formulated as a lyophilizate, or compounds described herein may be encapsulated within liposomes using technology known in the art. The pharmaceutical compositions may further comprise at least one additional pharmaceutical acceptable ingredient, which may be biologically active or inactive. Non-limiting examples of such ingredients include buffers (e.g., neutral buffered saline or phosphate buffered saline), carbohydrates (e.g., glucose, mannose, sucrose or dextrans), mannitol, proteins, polypeptides, amino acids (e.g., glycine), antioxidants, chelating agents (e.g., EDTA and glutathione), stabilizers, dyes, flavoring agents, suspending agents, and preservatives.

Any suitable excipient or carrier known to those of ordinary skill in the art for use in pharmaceutical compositions may be employed in the compositions described herein. Excipients for therapeutic use are well known, and are described, for example, in Remington: The Science and Practice of Pharmacy (Gennaro, 21^{st} Ed. Mack Pub. Co., Easton, PA (2005)). In general, the type of excipient is selected based on the mode of administration, as well as the chemical composition of the active ingredient(s). Pharmaceutical compositions may be formulated for the particular mode of administration. For parenteral administration, pharmaceutical compositions may further comprise water, saline, alcohols, fats, waxes, and buffers. For oral administration, pharmaceutical compositions may further comprise at least one ingredient chosen, for example, from any of the aforementioned excipients, solid excipients and carriers, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose, and magnesium carbonate.

The pharmaceutical compositions (e.g., for oral administration or delivery by injection) may be in the form of a liquid. A liquid pharmaceutical composition may include, for example, at least one of the following: a sterile diluent such as water for injection, saline solution, physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. In some embodiments, the pharmaceutical composition comprises physiological saline. In some embodiments, the pharmaceutical composition an injectable pharmaceutical composition, and in some embodiments, the injectable pharmaceutical composition is sterile.

For oral formulations, at least one of the compounds of the present disclosure can be used alone or in combination with at least one additive appropriate to make tablets, powders, granules, and/or capsules, for example, those chosen from conventional additives, disintegrators, lubricants, diluents, buffering agents, moistening agents, preservatives, coloring agents, and flavoring agents. The pharmaceutical compositions may be formulated to include at least one buffering agent, which may provide for protection of the active ingredient from low pH of the gastric environment and/or an enteric coating. A pharmaceutical composition may be formulated for oral delivery with at least one flavoring agent, e.g., in a liquid, solid or semi-solid formulation and/or with an enteric coating.

Oral formulations may be provided as gelatin capsules, which may contain the active compound or biological along with powdered carriers. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract.

A pharmaceutical composition may be formulated for sustained or slow release. Such compositions may generally be prepared using well known technology and administered by, for example, oral, rectal or subcutaneous implantation, or by implantation at the desired target site. Sustained-release formulations may contain the active therapeutic dispersed in a carrier matrix and/or contained within a reservoir surrounded by a rate controlling membrane. Excipients for use within such formulations are biocompatible and may also be biodegradable. The formulation may provide a relatively constant level of active component release. The amount of active therapeutic contained within a sustained release formulation depends upon the site of implantation, the rate and expected duration of release, and the nature of the condition to be treated or prevented.

The pharmaceutical compositions described herein can be formulated as suppositories by mixing with a variety of bases such as emulsifying bases or water-soluble bases. The pharmaceutical compositions may be prepared as aerosol formulations to be administered via inhalation. The compositions may be formulated into pressurized acceptable propellants such as dichlorodifluoromethane, propane, nitrogen, and the like.

The compounds of the present disclosure and pharmaceutical compositions comprising these compounds may be administered topically (e.g., by transdermal administration). Topical formulations may be in the form of a transdermal patch, ointment, paste, lotion, cream, gel, and the like. Topical formulations may include one or more of a penetrating agent or enhancer (also call permeation enhancer), thickener, diluent, emulsifier, dispersing aid, or binder. Physical penetration enhancers include, for example, electrophoretic techniques such as iontophoresis, use of ultrasound (or “phonophoresis”), and the like. Chemical penetration enhancers are agents administered either prior to, with, or immediately following administration of the therapeutic, which increase the permeability of the skin, particularly the stratum corneum, to provide for enhanced penetration of the drug through the skin. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneras et al., J. Pharm. Pharmacol. 54:499-508 (2002); Karande et al., Pharm. Res. 19:655-60 (2002); Vaddi et al., Int. J. Pharm. 91:1639-51 (2002); Ventura et al., J. Drug Target 9:379-93 (2001); Shokri et al., Int. J. Pharm. 228(1-2):99-107 (2001); Suzuki et al., Biol. Pharm. Bull. 24:698-700 (2001); Alberti et al., J. Control Release 71:319-27 (2001); Goldstein et al., Urology 57:301-5 (2001); Kiijavainen et al., Eur. J. Pharm. Sci. 10:97-102 (2000); and Tenjarla et al., Int. J. Pharm. 192:147-58 (1999).

Kits comprising unit doses of at least one compound of the present disclosure, for example in oral or injectable doses, are provided. Such kits may include a container comprising the unit dose, an informational package insert describing the use and attendant benefits of the therapeutic in treating the pathological condition of interest, and/or optionally an appliance or device for delivery of the at least one compound or composition comprising the same.

### EXAMPLES

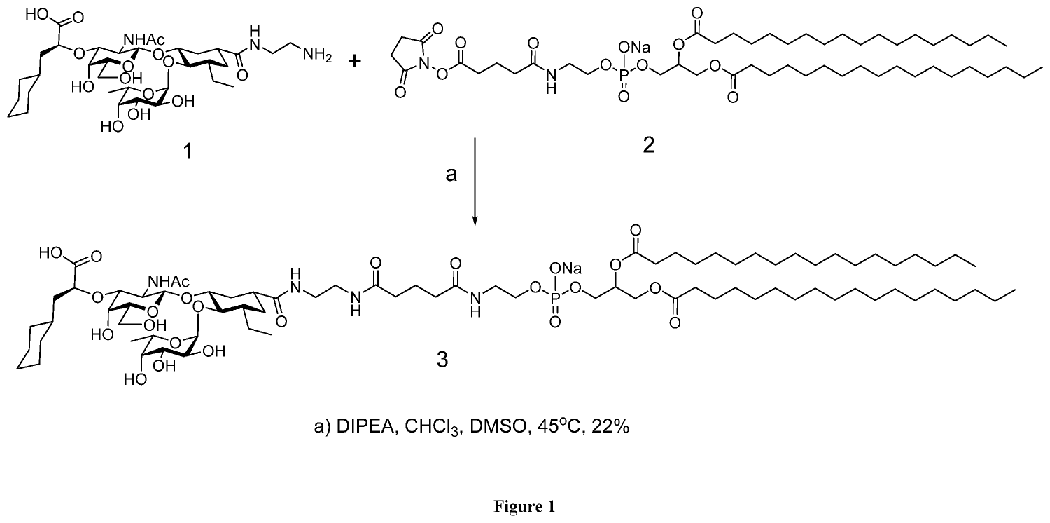

Compounds of Formula (I) may be prepared as shown in, for example, FIGS. 1-4. It is understood that one of ordinary skill in the art may be able to make these compounds by similar methods or by combining other methods known to one of ordinary skill in the art. It is also understood that one of ordinary skill in the art would be able to make other compounds of Formula (I) not specifically illustrated herein by using appropriate starting components and modifying the parameters of the synthesis as needed. In general, starting components may be obtained from sources such as Sigma Aldrich, Alfa Aesar, Maybridge, Matrix Scientific, TCI, and Fluorochem USA, etc. and/or synthesized according to sources known to those of ordinary skill in the art (see, for example, Advanced Organic Chemistry: Reactions, Mechanisms, and Structure, 5th edition (Wiley, December 2000)) and/or prepared as described herein.

It will also be appreciated by those skilled in the art that, in the processes described herein, the functional groups of intermediate compounds may need to be protected by suitable protecting groups, even if not specifically described. Such functional groups include hydroxy, amino, mercapto, and carboxylic acid. Suitable protecting groups for hydroxy include but are not limited to trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino, and guanidino include but are not limited to t-butoxycarbonyl, benzyloxycarbonyl, and the like. Suitable protecting groups for mercapto include but are not limited to —C(O)R″ (where R″ is alkyl, aryl, or arylalkyl), p-methoxybenzyl, trityl and the like. Suitable protecting groups for carboxylic acid include but are not limited to alkyl, aryl, or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin, or a 2-chlorotrityl-chloride resin.

Analogous reactants to those described herein may be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002.

Methods known to one of ordinary skill in the art may be identified through various reference books, articles, and databases. Suitable reference books and treatise that detail the synthesis of reactants useful in the preparation of compounds of the present disclosure, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure,” 4th Ed., Wiley-Interscience, New York, 1992. Additional suitable reference books and treatise that detail the synthesis of reactants useful in the preparation of compounds of the present disclosure, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

### Example 1

### Synthesis of Compound 3

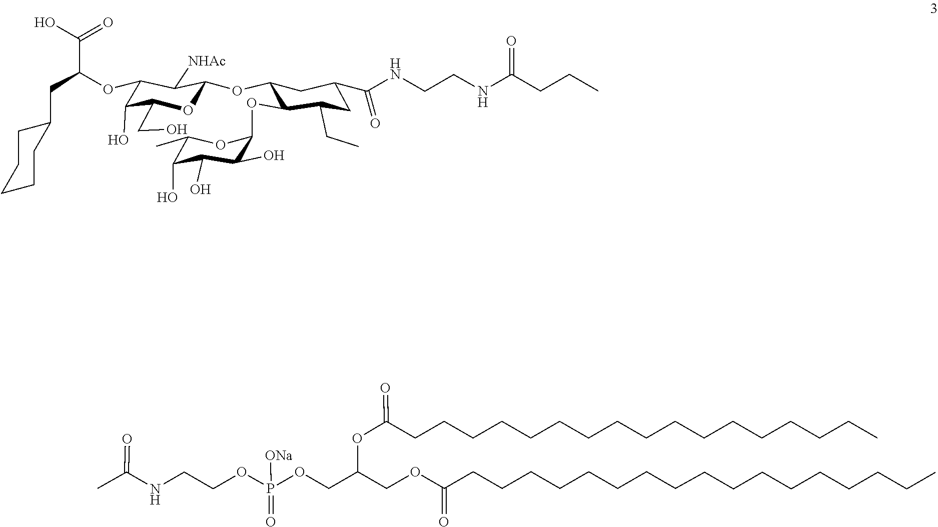

Compound 3: To a solution of 1,2-distearoyl-sn-glycero-3-phosphoethanolamine-NHS ester (DSPE-NHS, 62 mg, 62 μmol) in chloroform (0.4 mL) was added a solution of compound 1 (preparation described in WO2013/096926) (48 mg, 65 μmol) and one drop of DIPEA in DMSO (1 mL) over 5 minutes period at room temperature. The resulting solution was stirred overnight at 45° C. The solution was concentrated, and the residue was dried under high vacuum to remove residual solvent. The crude compound was purified by normal phase silica chromatography eluting with EtOAc/MeOH, 2/1-MeOH to provide compound 3 (22 mg, 14 μmole, 22%). HPLC purity: 98.8%, LC-MS: 1576.8 (M−1).

¹H NMR (400 MHz, Methanol-d₄, CDCl₃) δ 7.62-7.53 (m, 2H), 3.91 (d, J=12.7 Hz, 1H), 3.79-3.69 (m, 1H), 3.48-3.39 (m, 1H), 3.36 (d, J=5.2 Hz, 9H), 2.29 (dt, J=27.0, 9.8 Hz, 2H), 2.05-1.97 (m, 1H), 1.64-1.59 (m, 1H), 1.32-1.18 (m, 14H), 0.89 (dq, J=17.6, 7.0, 5.6 Hz, 3H).

### Example 2

### Prophetic Synthesis of Compound 5

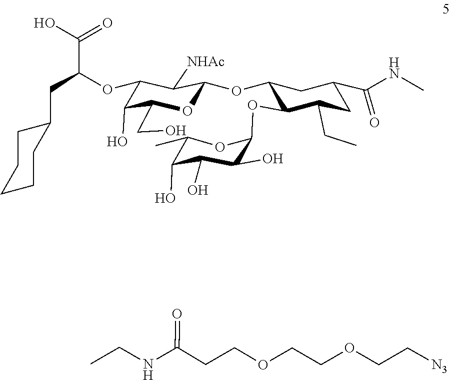

Compound 5: Compound 1 (1 equivalent) and azido PEG NHS ester compound 4 (1.2 equivalents) are combined in water with triethylamine (1.5 equivalents). The reaction mixture is stirred at room temperature until completion. The reaction mixture is neutralized by the addition of acetic acid and the mixture separated by HPLC to afford compound 5.

### Example 3

### Prophetic Synthesis of Compound 6

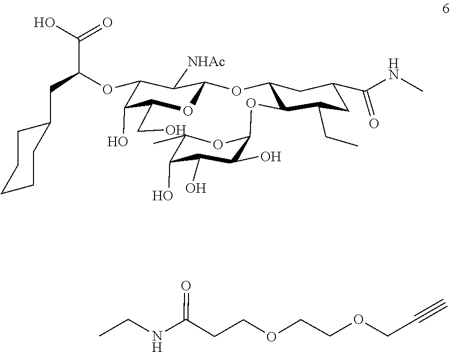

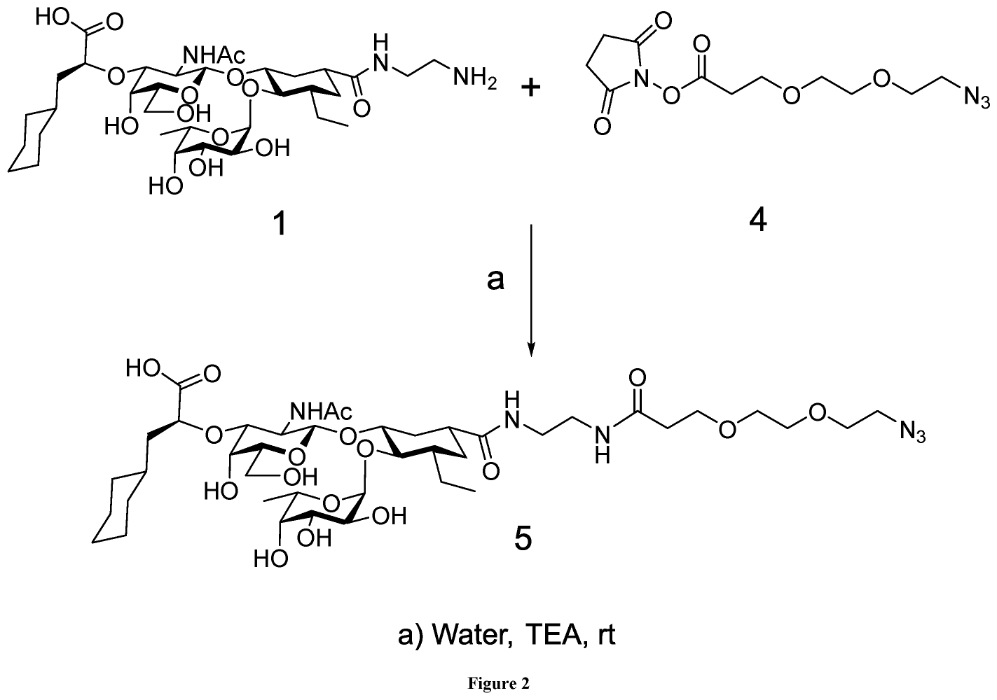

Compound 6: Compound 6 can be prepared according to FIG. 2 by using a propargyl PEG NHS ester in place of compound 4.

### Example 4

### Prophetic Synthesis of Compound 7

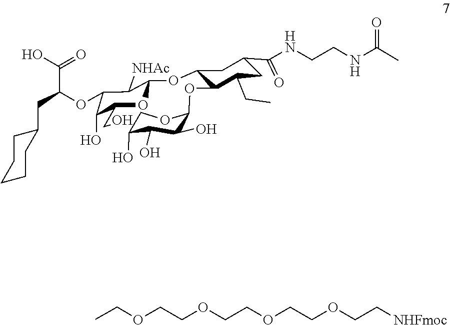

Compound 7: Compound 7 can be prepared according to FIG. 2 by using an Fmoc-amino PEG NHS ester in place of compound 4.

### Example 5

### Prophetic Synthesis of Compound 8

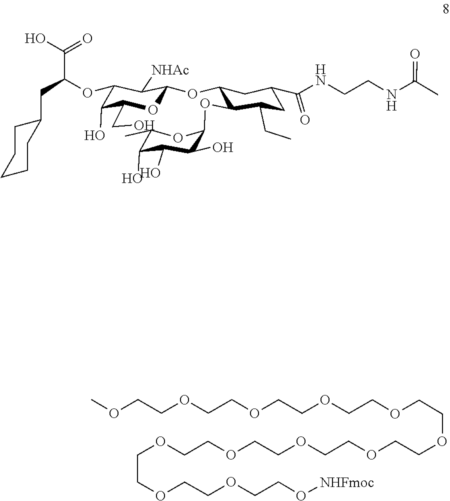

Compound 8: Compound 8 can be prepared according to FIG. 2 by using an Fmoc-amino-oxy PEG NHS ester in place of compound 4.

### Example 6

### Prophetic Synthesis of Compound 9

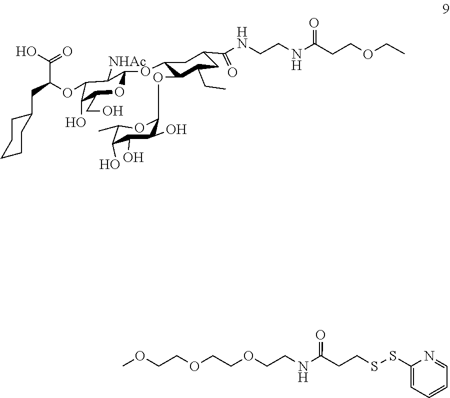

Compound 9: Compound 9 can be prepared according to FIG. 2 by using an SPDP PEG NHS ester in place of compound 4.

### Example 7

### Prophetic Synthesis of Compound 10

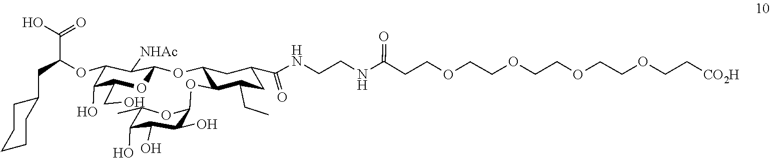

Compound 10: Compound 10 can be prepared according to FIG. 2 by using a carboxy PEG NHS ester in place of compound 4.

### Example 8

### Prophetic Synthesis of Compound 11

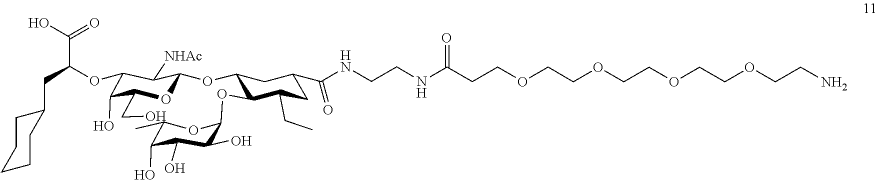

Compound 11: Compound 7 (1 equivalent) is dissolved in water. Piperidine (2 equivalents) is added. The reaction mixture is stirred at room temperature until completion. Compound 11 is isolated by HPLC.

### Example 9

### Prophetic Synthesis of Compound 12

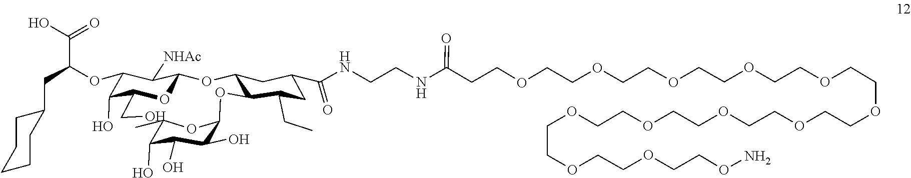

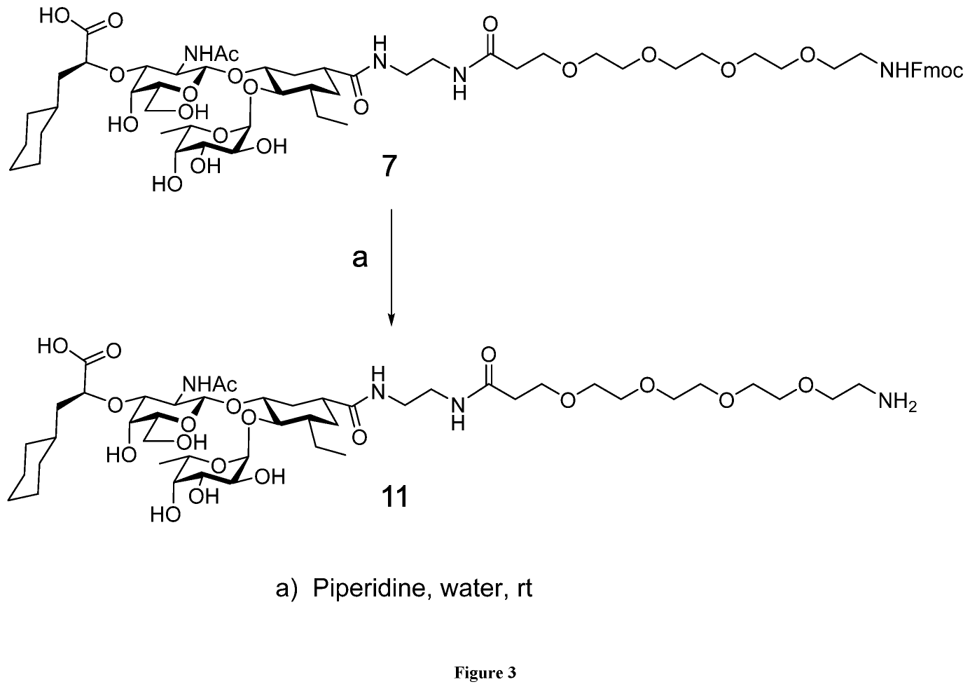

Compound 12: Compound 12 is prepared according to FIG. 3 using the same procedure as for the preparation of compound 11.

### Example 10

### Prophetic Synthesis of Compound 13

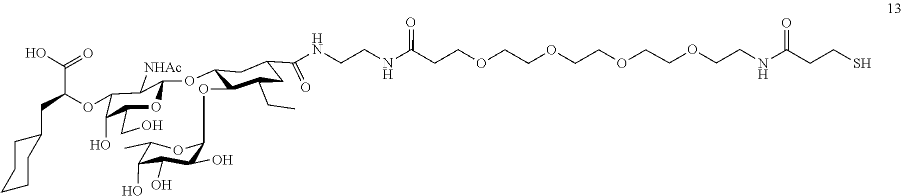

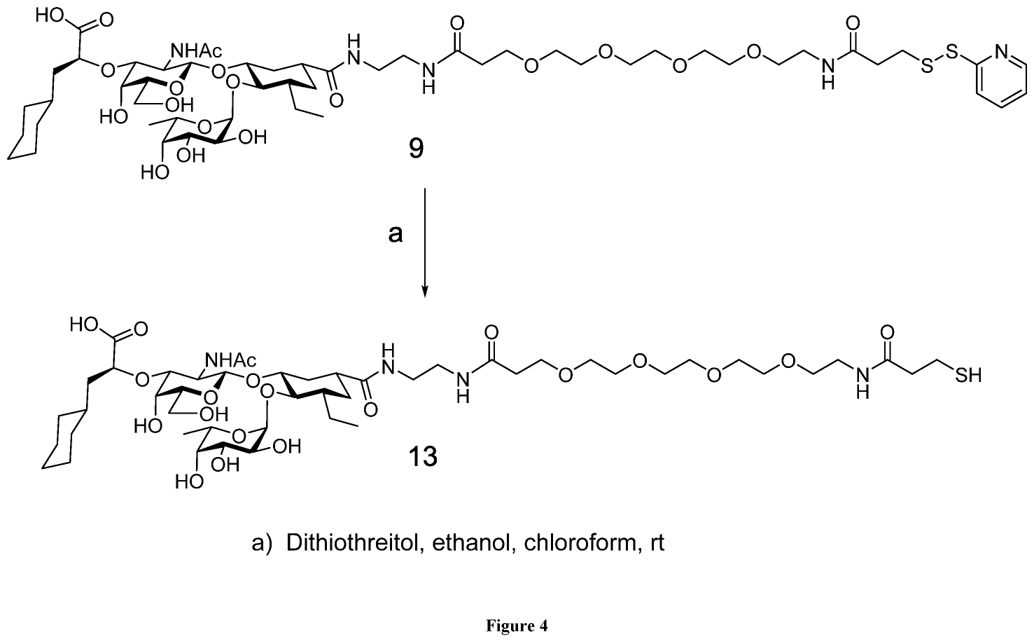

Compound 13: As shown in FIG. 4: Compound 9 is dissolved ethanol/chloroform and degassed by bubbling argon through the solution for 5 minutes. Dithiothreitol (3 equivalents) is added and the reaction mixture is stirred at room temperature until completion. The solvent is removed, and the residue is separated by HPLC to afford compound 13.

### Example 11

### Liposome-Incorporation of Compound 3

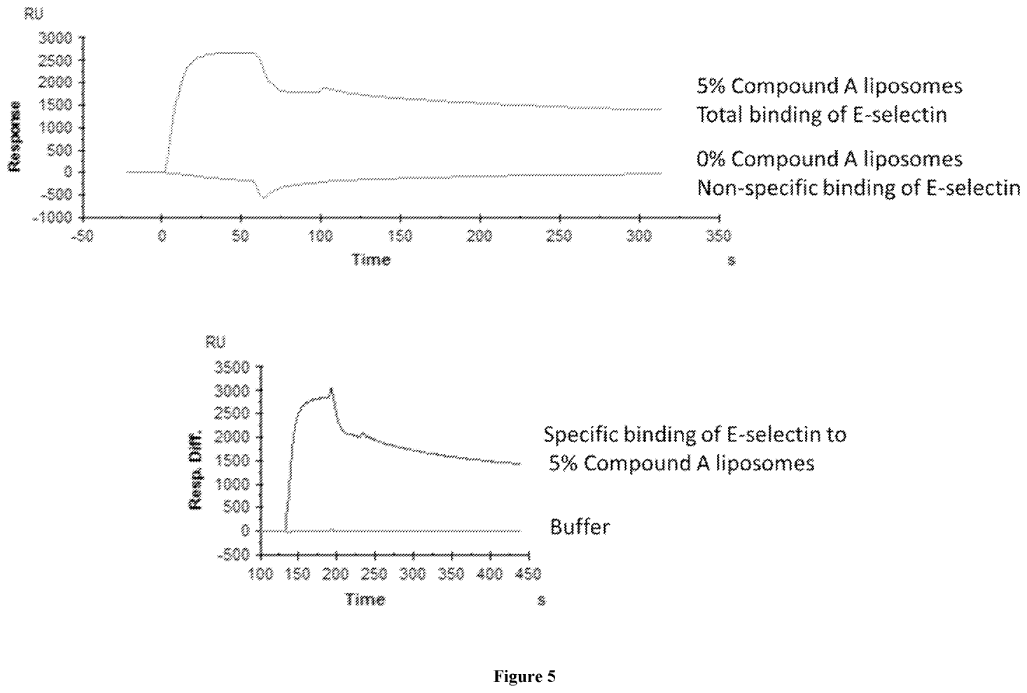

Compound 3 (referred to as “compound A” in FIGS. 5, 6, and 8) was incorporated into unilamellar synthetic phospholipid vesicles (liposomes) composed of high phase-transition-temperature (Tm) phospholipid hydrogenated soy phosphatidylcholine hydrogenated phosphatidylcholine (HSPC)/cholesterol (CHOL)/1,2-Distearoyl-sn-glycero-3-phosphoethanolamine (DSPE) in a molar ratio of 56:38:5 using established techniques. For liposomes containing the E-selectin targeting agent, DSPE was replaced with the identical 5% molar ratio of Compound A (Table 1). In one embodiment, a trace amount of fluorescein-PE was incorporated into the liposomes as a membrane label. Briefly, lipid stocks prepared in chloroform were combined to achieve the desired mole percentage (mol %) of each (final lipid amount of 10 mmol). Following solvent removal by rotary evaporation and vacuum, the resulting lipid shells were hydrated and sonicated in phosphate buffered saline (PBS) at 80° C. to a final lipid concentration of 1 mM. The resulting multilamellar vesicles underwent three cycles of freeze/thaw and unilamellar vesicles were generated by extrusion through a 100 nm pore membrane.

Four preparations of liposomes were manufactured as summarized in Table 1. In some preparations a trace amount of fluorescent phosphatidylethanolamine was included to allow specific experimentation with the liposomes.

TABLE 1
Lipid composition of liposomes containing Compound 3.

| | MOL % | | | |
|---|---|---|---|---|
| | Compo- | Compo- | Compo- | Compo- |
| LIPID | sition 1 | sition 2 | sition 3 | sition 4 |
| HSPS | 55 | 55 | 55 | 55 |
| Cholesterol | 40 | 40 | 40 | 40 |
| DSPE | 5 | 0 | 5 | 0 |
| Compound 3 | 0 | 5 | 0 | 5 |
| Fluorescein-PE | 0 | 0 | 0.2 | 0.2 |

### Example 12

### Demonstration of Specific Targeting of E-Selectin and E-Selectin Expressing Endothelial Cells

Surface Plasmon Resonance (SPR) studies were conducted wherein liposome preparations L1 and L3 were captured (6,000-10,000 resonance units; RU) on a lipophilic sensor chip at a flow rate of 5 mL/min in PBS/0.5% bovine serum albumin (BSA)/2 mM CaCl₂) as running buffer. Running buffer alone or E-selectin-Fc chimera (0.1 mg/ml; R&D Systems) was then injected at a flow rate of 5 μL/min in running buffer and the response was recorded. The results demonstrate that only those liposomes with incorporated Compound 3 (referred to as “compound A” in FIGS. 5, 6 and 8) had the capacity to bind E-selectin. No interaction with E-selectin was observed when liposomes did not contain Compound 3. The results of this study are summarized in FIG. 5.

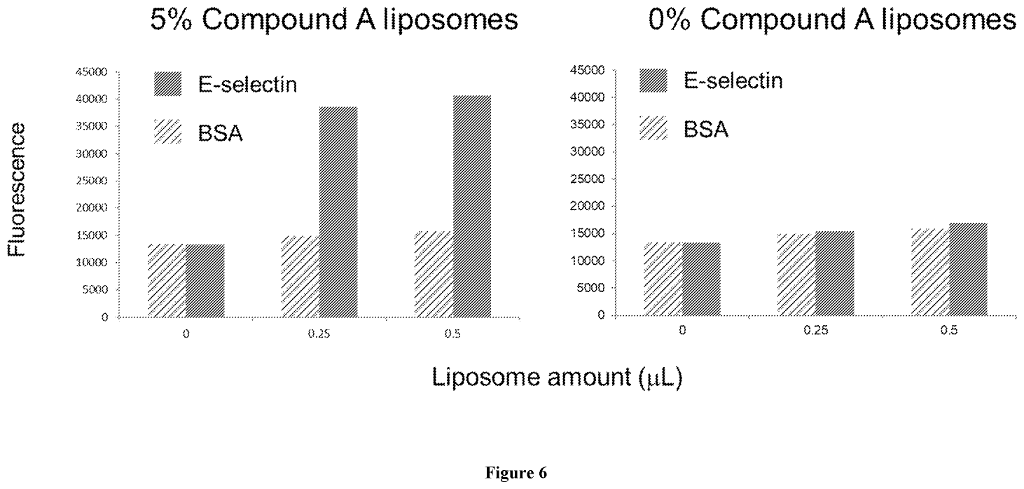

A fluorescent-based plate assay was used to further investigate the specificity of E-selectin targeting with liposomes containing Compound 3. E-selectin-Fc chimera or BSA were adsorbed onto wells of a 96-well plate after which liposome preparation L4 or L5 were added and allowed to adhere for 15 min at 37° C. The wells were washed 3× and the amount of fluorescence liposomes bound to each substrate was determined. The results demonstrate that only those liposomes with incorporated Compound 3 had the capacity to bind E-selectin. Background binding to BSA was comparable with both liposome preparations and again demonstrating the targeting capacity of liposomes containing Compound 3 for E-selectin. The results of this study are summarized in FIG. 6.

Subsequent studies were performed to investigate the ability of liposomes containing Compound 3 to recognize and bind to cells under conditions where E-selectin is expressed. For these liposomes with and without Compound 3 were prepared with a fluorescent phospholipid membrane marker (Composition 4 and Composition 3, respectively) allowing for detection by flow cytometry. Liposomes were incubated for 60 min at 37° C. with cultures of human umbilical vein endothelial cells (HUVEC) pretreated in media deficient in TNFα or supplemented with TNFα to induce E-selectin expression. Association of fluorescein labeled liposomes with cell preparations by flow cytometry was then determined.

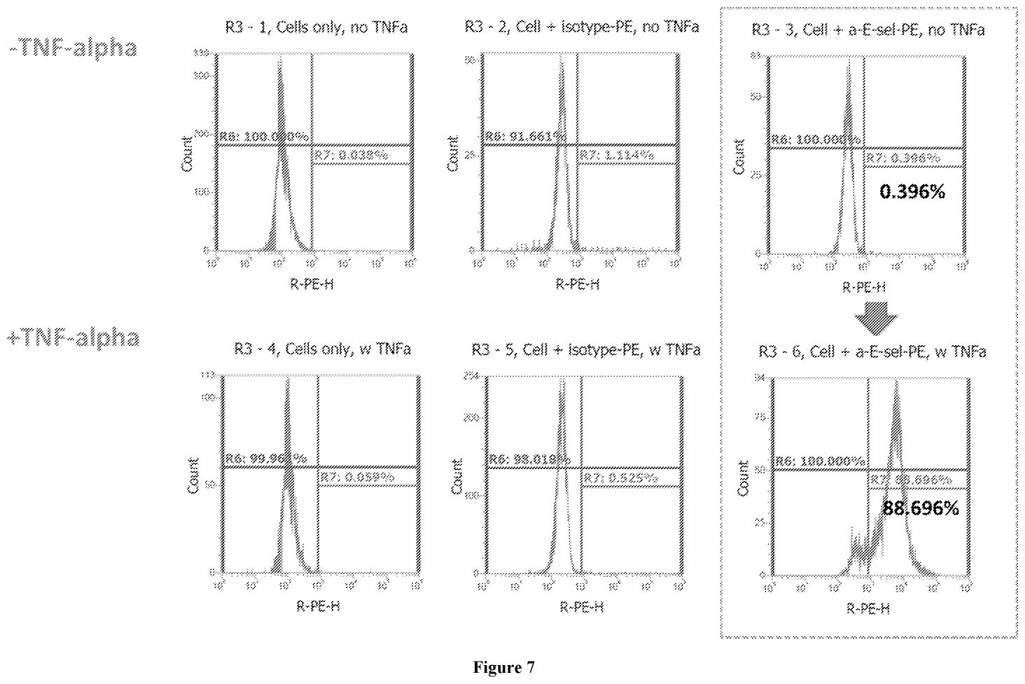

The flow cytometry-based assay used liposomes containing 0% or 5% Compound 3 (L4 and L5, respectively). Initial studies defined that only TNFα-stimulated HUVEC but not unstimulated HUVEC express E-selectin. Using the anti-E-selectin antibody clone HAE-if the results show that with TNFα stimulation 88.696% of HUVEC express E-selectin as compared to 0.396% unstimulated HUVEC. These results are summarized in FIG. 7.

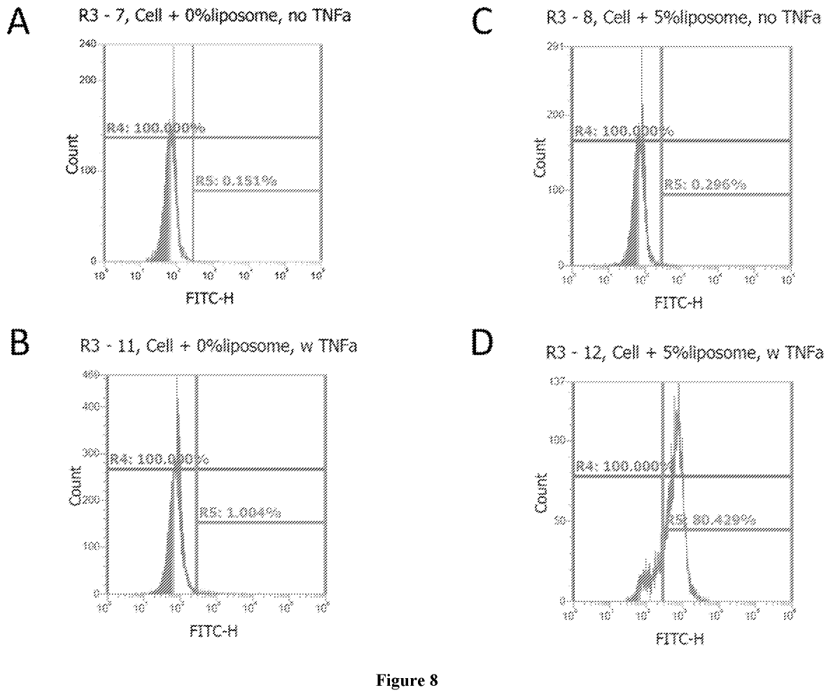

Using these culture conditions, unstimulated or TNFα-stimulated HUVEC were incubated with liposomes containing 0% or 5% Compound 3 (L4 and L5, respectively). The results demonstrated that only liposomes containing Compound 3 bind to TNFα-activated HUVEC, but not unstimulated HUVEC (80.429% vs. 0.296%, respectively). Control liposomes not containing Compound 3 fail to bind either activated or unstimulated HUVEC (1.004 vs 0.151%, respectively). These results are summarized in FIG. 8.

Collectively, these data demonstrate that the incorporation of Compound 3 into a liposomal formulation can generate a targeted carrier that is specific and selective for E-selectin and E-selectin expressing endothelial cells.

### Example 13

### Demonstration of Specific Targeting In Vivo

The purpose of this study was to provide quantitative assessment of the in vivo tumor localization of IRdye780-labeled liposomes containing Compound 3 and control IR780 labeled liposomes (no Compound 3) at 4 hours, 24 hours, and 1-week post-i.v. dosing using fluorescence imaging (FLI) on the IVIS® Lumina S5 imaging system (IVIS), in female C.B-17 SCID mice with established subcutaneous (SC) A549 human non-small cell lung cancer (NSCLC) tumors.

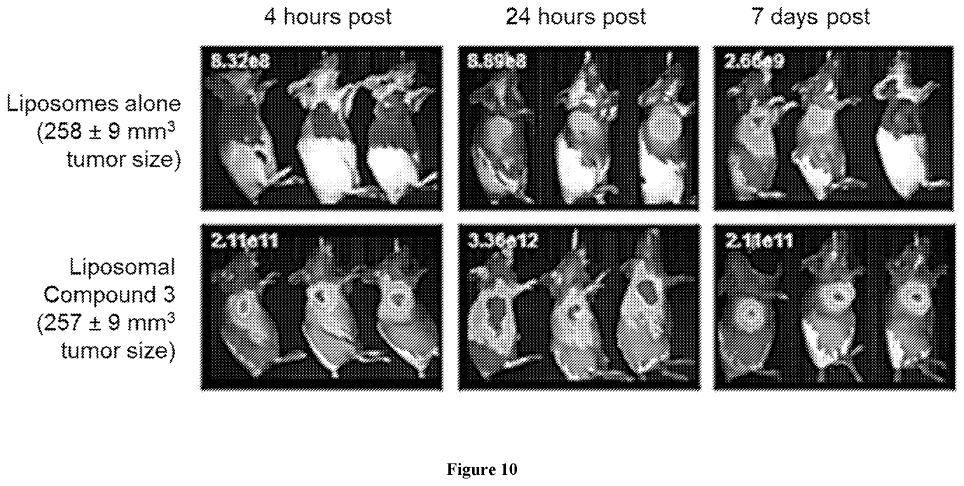

In vivo FLI images were acquired in a lateral position at 4 hours post, 24 hours post, and 1 week post liposome injection (FIG. 10, lateral view of right axillary tumor). FLI images were acquired using the excitation/emission filters (745/800).

All treatments were well tolerated. All mice were shaved/epilated 48 hours before dosing.

The FLI signal was localized in the tumor and the kinetics of the signal was the highest at 24 hours post dosing (mean=3.36×10¹² fluorescence intensity units) when compared to 4 hours (mean=2.11×10¹¹ fluorescence intensity units) and 1-week (mean=2.11×10¹¹ fluorescence intensity units) post injection time points in mice dosed with 2 nmol IRdye780-labeled liposomes containing Compound 3. Injection of mice with 2 nmol IRdye780-labeled liposomes only (no Compound 3) showed marginal tumor localization.

1. At least one entity chosen from compounds of Formula (I): 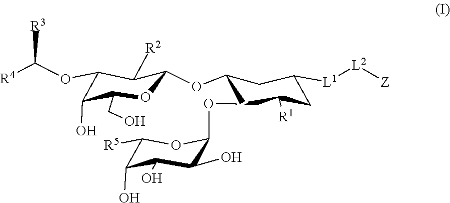
and pharmaceutically acceptable salts thereof, wherein
R¹ is chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, 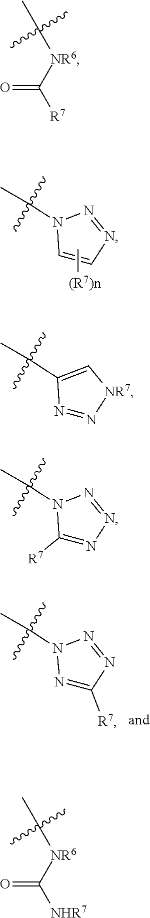
groups, wherein n is chosen from integers ranging from 0 to 2, R⁶ is chosen from H, C₁₋₁₂ alkyl, C₂₋₁₂ alkenyl, C₂₋₁₂ alkynyl, C₄₋₁₆ cycloalkylalkyl, and —C(═O)R⁷ groups, and each R⁷ is independently chosen from H, C₁₋₁₂ alkyl, C₂₋₁₂ alkenyl, C₂₋₁₂ alkynyl, C₄₋₁₆ cycloalkylalkyl, C₆₋₁₈ aryl, and C₁₋₁₃ heteroaryl groups;
R² is chosen from —OH, —OY¹, halo, —NH₂, —NHY¹, —NY¹Y², —OC(═O)Y¹, —NHC(═O)Y¹, —NHC(═O)NHY¹, and —NHC(═O)NY¹Y² groups, wherein Y¹ and Y², which may be the same or different, are independently chosen from C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₄₋₁₆ cycloalkylalkyl, C₂₋₁₂ heterocyclyl, C₆₋₁₅ aryl, and C₁₋₁₃ heteroaryl groups, or Y¹ and Y² may join together along with the heteroatom to which they are attached to form an optionally substituted, saturated or unsaturated ring;
R³ is chosen from —CN, —CH₂CN, —C(═O)Y³, —C(═O)OH, —C(═O)OY³, —C(═O)NH₂, —C(═O)NHOH, —C(═O)NHOCH₃, —NHCN, —C(═O)NHY³, —C(═O)NY³Y⁴, —S(═O)₂Y³, —S(═O)₂₀Y³, —S(═O)₂NH₂, —S(═O)₂NHY³, and —S(═O)₂NY³Y⁴ groups, wherein Y³ and Y⁴, which may be identical or different, are independently chosen from C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₁₋₁₃ heterocyclyl, and C₇₋₁₂ arylalkyl groups, or Y³ and Y⁴ join together along with the heteroatom to which they are attached to form an optionally substituted, saturated or unsaturated ring;
R⁴ is chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₁₋₁₂ alkoxy, C₄₋₁₆ cycloalkylalkyl, C₆₋₁₈ aryl, and C₂₋₁₃ heteroaryl groups;
R⁵ is chosen from —CN, C₁₋₁₂ alkyl, and C₁₋₁₂ haloalkyl groups;
L¹ is chosen from 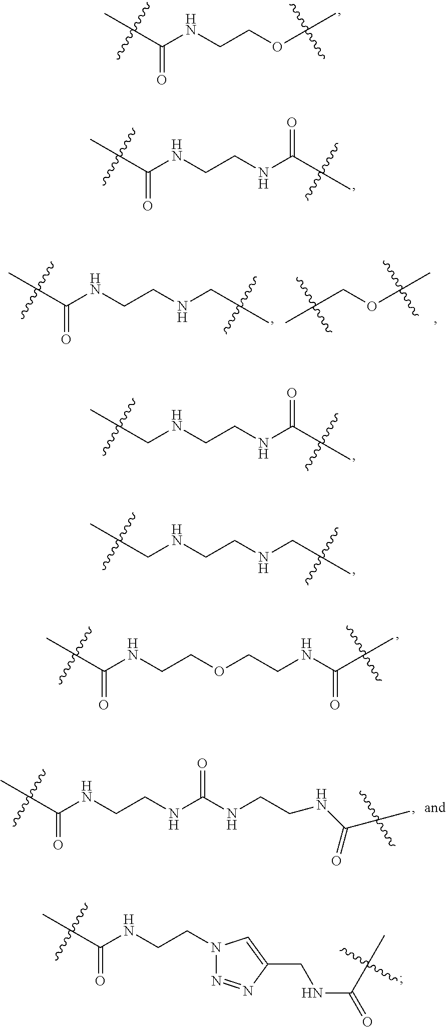
L² is chosen from —CH₂(OCH₂CH₂)ₘV—, —CH₂CH₂(OCH₂CH₂)ₘV—, and —(CH₂) mV— groups, wherein V is chosen from a bond, —O—, —OCH₂—, —NHC(═O)CH₂CH₂—, and —C(═O)NHCH₂CH₂—, and wherein m is chosen from integers ranging from 1 to 46; and
Z is chosen from lipids, nucleophiles, electrophiles, and groups capable of undergoing a cycloaddition reaction.

2. The at least one entity according to claim 1, wherein R¹ is chosen from C₁₋₄ alkyl,

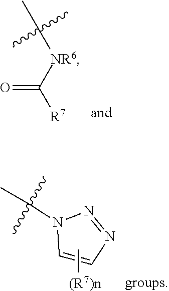

3. The at least one entity according to claim 2, wherein R¹ is chosen from methyl, ethyl,

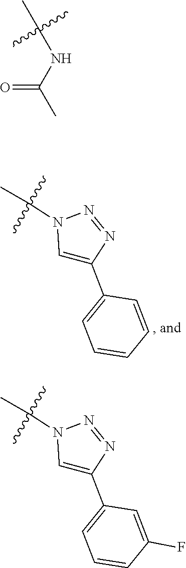

4. The at least one entity according to claim 3, wherein R¹ is ethyl.

5. The at least one entity according to claim 1, wherein R² is chosen from —OH, —OY¹, —OC(═O)Y¹, —NH₂, and —NHC(═O)Y¹ groups, wherein Y¹ is chosen from C₁₋₈ alkyl, C₄₋₁₆ cycloalkylalkyl, C₂₋₁₂ heterocyclyl, C₆₋₁₈ aryl, and C₁₋₁₃ heteroaryl groups.

6. The at least one entity according to claim 5, wherein R² is chosen from

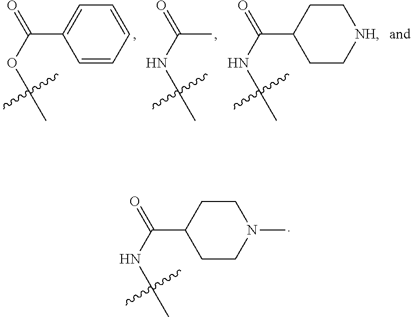

7. The at least one entity according to claim 6, wherein R² is

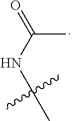 [SMILES: *NC(C)=O]

8. The at least one entity according to claim 1, wherein R³ is chosen from —C(═O)OH, —C(═O)OY³, —C(═O)NH₂, —C(═O)NHOH, —C(═O)NHOCH₃, —C(═O)NHY³, and —C(═O)NY³Y⁴ groups, wherein Y³ and Y⁴, which may be identical or different, are independently chosen from C₁₋₈ alkyl, C₁₋₈ haloalkyl, and C₇₋₁₂ arylalkyl groups, or Y³ and Y⁴ join together along with the heteroatom to which they are attached to form an optionally substituted, saturated or unsaturated ring.

9. The at least one entity according to claim 8, wherein R³ is chosen from

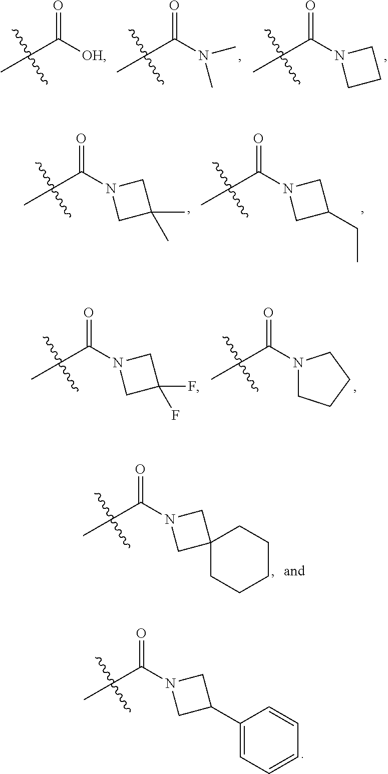

10. The at least one entity according to claim 9, wherein R³ is chosen from

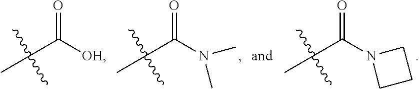

11. The at least one entity according to claim 10, wherein R³ is

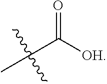 [SMILES: *C(=O)O]

12. The at least one entity according to claim 1, wherein R⁴ is chosen from C₁₋₈ alkyl and C₄₋₁₆ cycloalkylalkyl groups.

13. The at least one entity according to claim 12, wherein R⁴ is chosen from

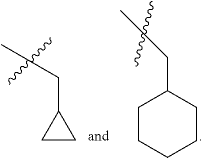 [SMILES: *CC1CC1 ; *CC1CCCCC1]

14. The at least one entity according to claim 13, wherein R⁴ is

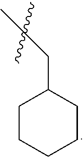 [SMILES: *CC1CCCCC1]

15. The at least one entity according to claim 1, wherein R⁵ is chosen from —CN, —CF₃, and methyl.

16. The at least one entity according to claim 15, wherein R⁵ is methyl.

17. The at least one entity according to claim 1, wherein L¹ is chosen from

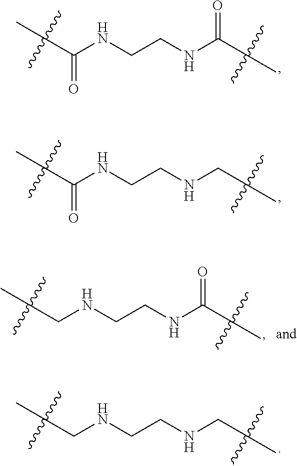 [SMILES: *C(=O)NCCNC(*)=O ; *CNCCNC(*)=O ; *CNCCNC(*)=O ; *CNCCNC*]

18. The at least one entity according to claim 17, wherein L¹ is

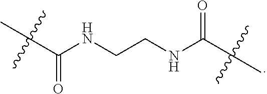 [SMILES: *C(=O)NCCNC(*)=O]

19. The at least one entity according to claim 1, wherein L² is chosen from —(CH₂)ₘV— groups, wherein V is —(═O)NHCH₂CH₂—, and m is chosen from integers ranging from 1 to 10.

20. The at least one entity according to claim 19, wherein m is 3.

21. The at least one entity according to claim 1, wherein L² is chosen from —CH₂(OCH₂CH₂)ₘV— groups, wherein V is chosen from a bond, —O—, and —OCH₂—, and m is chosen from integers ranging from 1 to 20.

22. The at least one entity according to claim 21, wherein m is chosen from integers ranging from 1 to 6.

23. The at least one entity according to claim 21, wherein L² is —CH₂OCH₂CH₂—.

24. The at least one entity according to claim 21, wherein L² is —CH₂(OCH₂CH₂)₂—.

25. The at least one entity according to claim 21, wherein L² is —CH₂(OCH₂CH₂)₄—.

26. The at least one entity according to claim 21, wherein L² is —CH₂(OCH₂CH₂)₆O—.

27. The at least one entity according to claim 21, wherein L² is —CH₂(OCH₂CH₂)₆OCH₂—.

28. The at least one entity according to claim 1, wherein L² is chosen from —CH₂CH₂(OCH₂CH₂)ₘV— groups, wherein V is chosen from a bond, —O—, —OCH₂—, and —NHC(═O)CH₂CH₂—, and m is chosen from integers ranging from 1 to 30.

29. The at least one entity according to claim 28, wherein m is chosen from integers ranging from 1 to 14.

30. The at least one entity according to claim 28, wherein L² is —CH₂CH₂(OCH₂CH₂)₂—.

31. The at least one entity according to claim 28, wherein L² is —CH₂CH₂(OCH₂CH₂)OCH₂—.

32. The at least one entity according to claim 28, wherein L² is —CH₂CH₂(OCH₂CH₂)₄—.

33. The at least one entity according to claim 28, wherein L² is —CH₂CH₂(OCH₂CH₂)₄NH(═O)CH₂CH₂—.

34. The at least one entity according to claim 28, wherein L² is —CH₂CH₂(OCH₂CH₂)₁₂O—.

35. The at least one entity according to claim 1, wherein Z is chosen from —N₃, —NH₂, —N(OH)H, —SH, —OH, —Cl, —Br, —I, —CH═CH₂, —C≡CH,

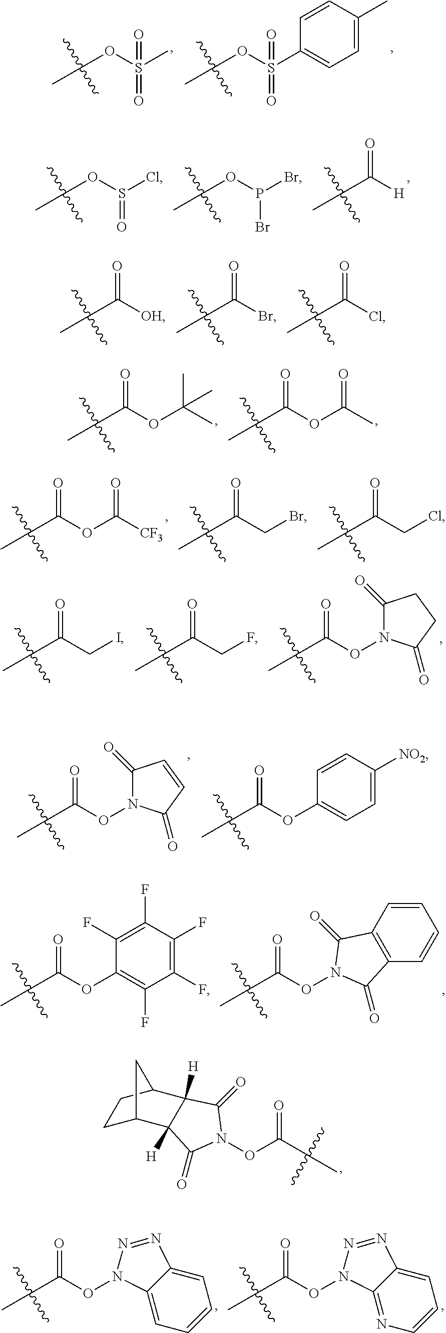

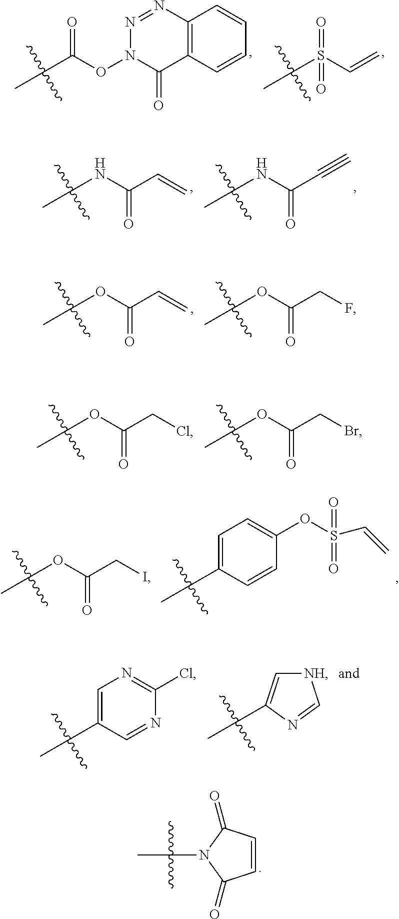

36. The at least one entity according to claim 35, wherein Z is —N₃.

37. The at least one entity according to claim 35, wherein Z is chosen from —NH₂, —SH, and —OH.

38. The at least one entity according to claim 35, wherein Z is chosen from —C(═O)H, —C(═O)OH, —C(═O)Cl, and —C(═O)O^{t}Bu.

39. The at least one entity according to claim 1, wherein Z is chosen from esters formed with t-butanol, p-nitrophenol, 2,4-dinitrophenol, trichlorophenol, 1-hydroxy-1H-benzotriazole, 1-hydroxy-6-chloro-1H-benzotriazole, and N-hydroxysuccinimide.

40. The at least one entity according to claim 1, wherein Z is chosen from —C(═O)Y⁵ groups, wherein Y⁵ is chosen from H, —OH, —O^{t}Bu, C₂₋₈ alkenyl, C₁₋₈ haloalkyl, C₁₋₈ alkoxy, halo, C₆₋₁₈ aryloxy, C₁₋₁₃ heteroaryloxy, and C₂₋₁₂ heterocyclyloxy groups and wherein the C₁₋₈ alkoxy, C₆₋₁₈ aryloxy, C₁₋₁₃ heteroaryloxy, and C₂₋₁₂ heterocyclyloxy groups are optionally substituted with at least one halo group.

41. The at least one entity according to claim 1, wherein Z is chosen from alkene and alkyne groups.

42. The at least one entity according to claim 41, wherein Z is —C≡CH.

43. The at least one entity according to claim 1, wherein Z is chosen from lipids.

44. The at least one entity according to claim 43, wherein Z is chosen from phospholipids.

45. The at least one entity according to claim 43, wherein Z is

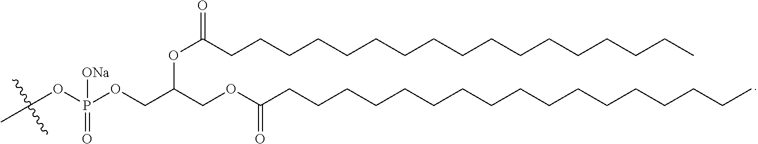

46. The at least one entity according to claim 1, wherein the entity is chosen from: 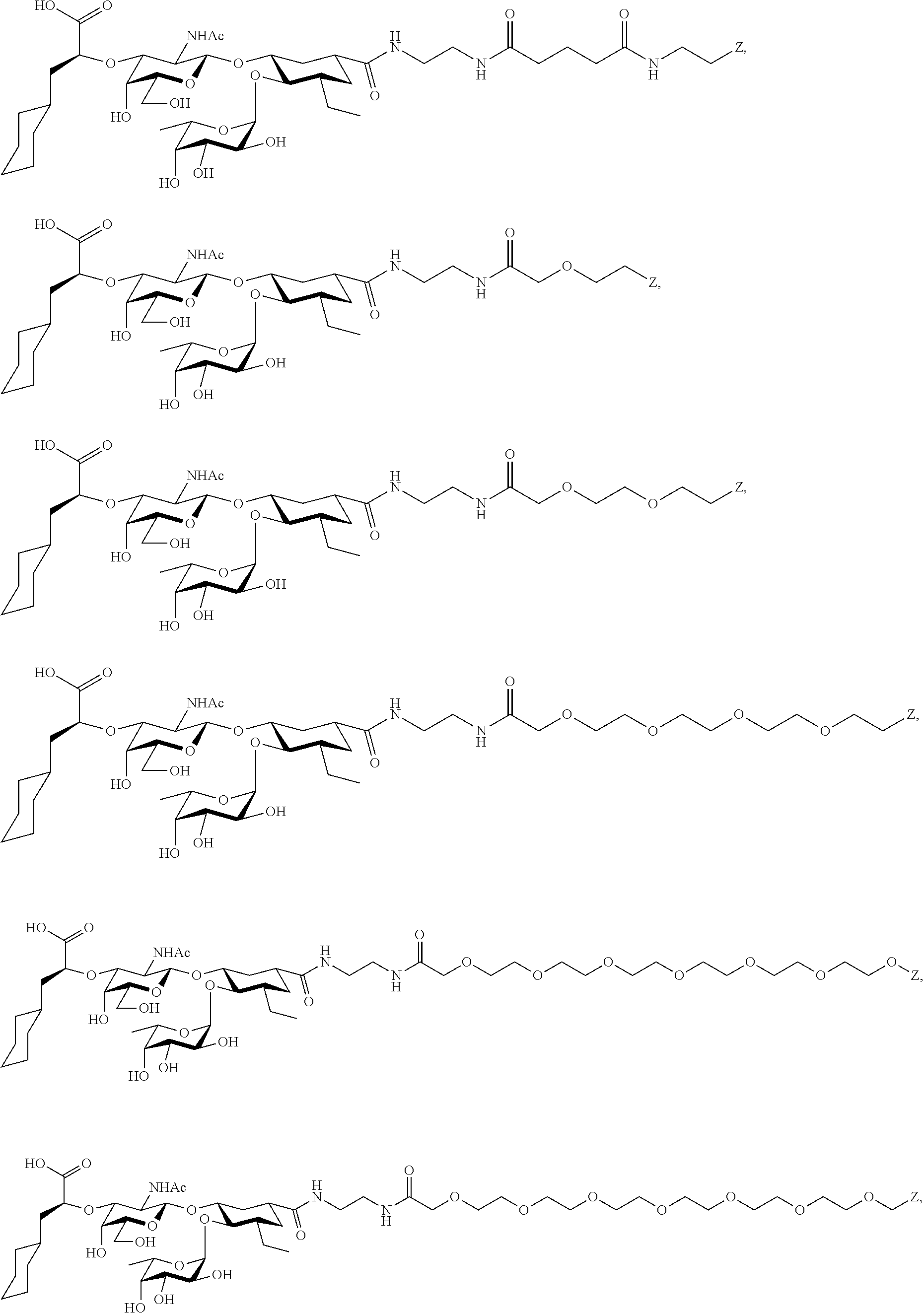 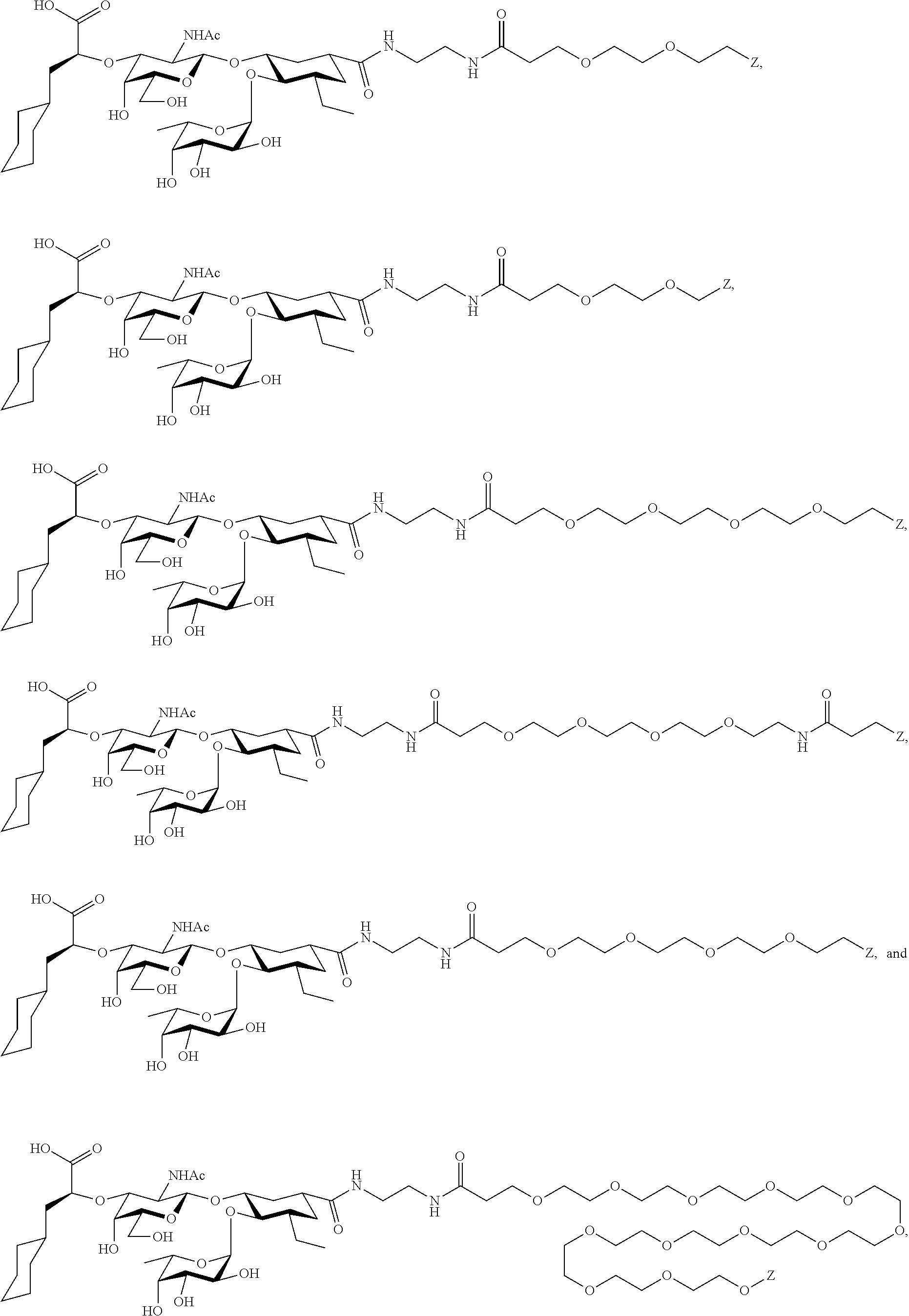
and pharmaceutically acceptable salts of any of the foregoing.

47. A process for making at least one entity chosen from compounds of Formula (II), prodrugs of compounds of Formula (II), and pharmaceutically acceptable salts of any of the foregoing, wherein the process comprises reacting or associating at least one entity of claim 1, or a protected form thereof, with at least one entity chosen from compounds of Formula (III): 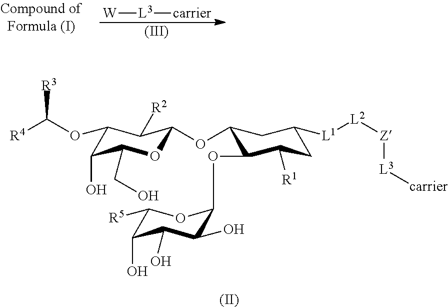
wherein R¹, R², R³, R⁴, R⁵, L¹, and L² are as defined in claim 1;
L³ is chosen from a bond and linker groups;
W is chosen from lipids, nucleophiles, electrophiles, and groups capable of undergoing a cycloaddition reaction; and
Z′ is a moiety generated by the reaction or association of the Z group of the at least entity of claim 1 with the W group of the at least one entity chosen from compounds of Formula (III).

48. The process according to claim 47, wherein the at least one entity of claim 1 is chosen from: 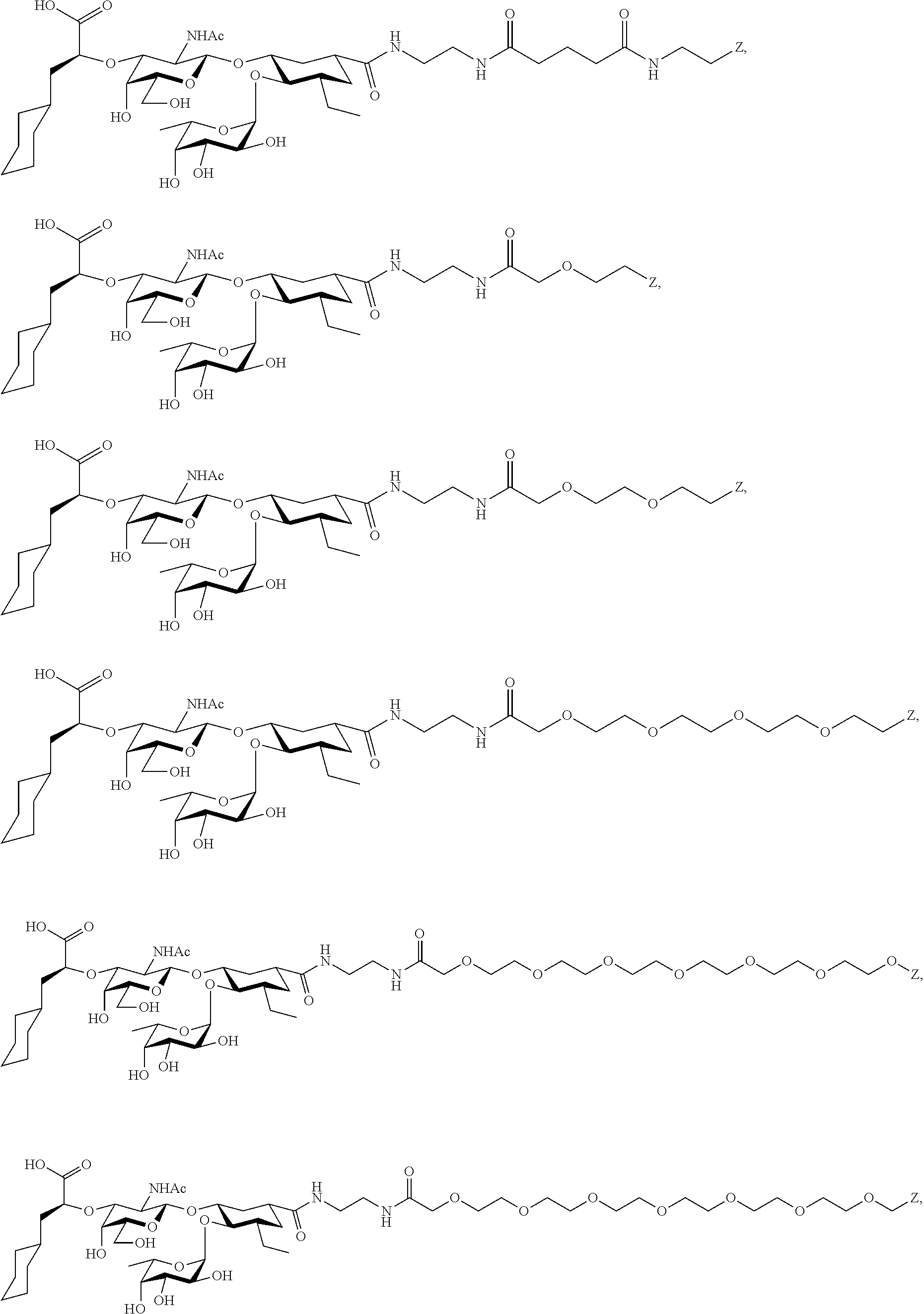 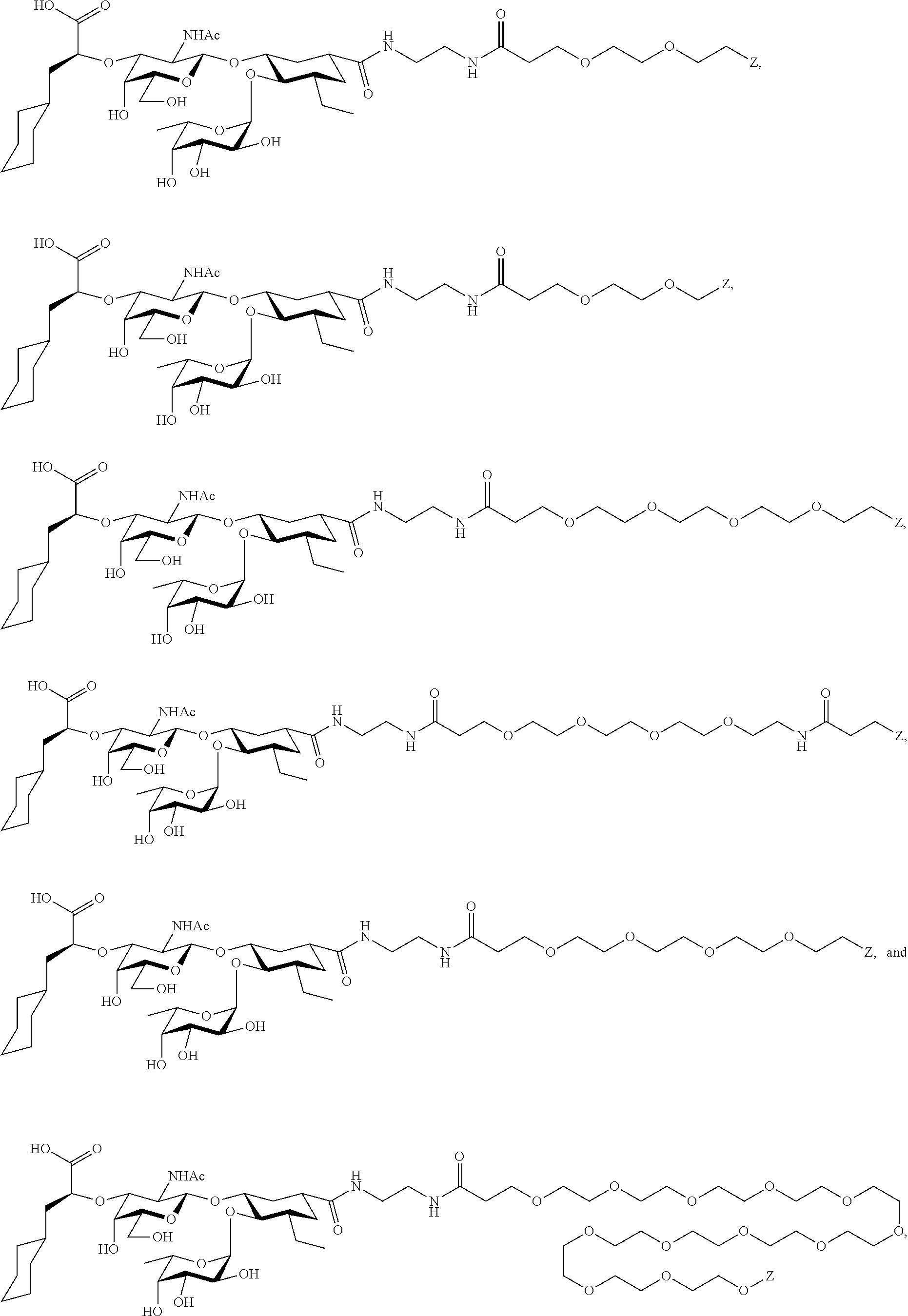
and pharmaceutically acceptable salts of any of the foregoing.

49. The process according to claim 47, wherein L³ is a bond.

50. The process according to claim 47, wherein L³ is chosen from linker groups.

51. The process according to claim 50, wherein the linker groups are chosen from

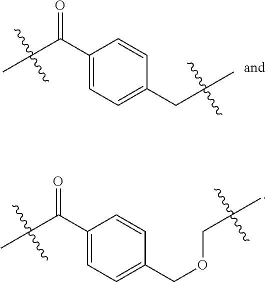 [SMILES: *Cc1ccc(C(*)=O)cc1 ; *COCc1ccc(C(*)=O)cc1]

52. The process according to claim 47, wherein the carrier is chosen from particles, nanoparticles, liposomes, beads, proteins, polysaccharides, lipids, metal chelating agents, and combinations of any of the foregoing.

53. The process according to claim 52, wherein the carrier is chosen from nanoparticles.

54. The process according to claim 52, wherein the carrier is chosen from lipids.

55. The process according to claim 54, wherein the lipids are chosen from phospholipids.

56. The process according to claim 52, wherein the carrier is chosen from metal chelating agents.

57. The process according to claim 56, wherein the metal chelating agents are chosen from:

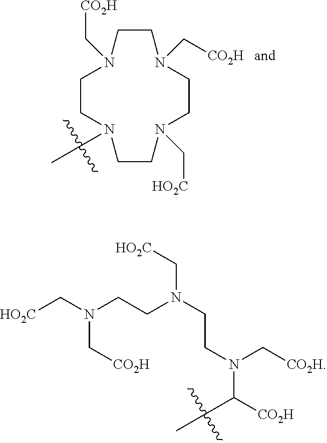 [SMILES: *N1CCN(CC(=O)O)CCN(CC(=O)O)CCN(CC(=O)O)CC1 ; *C(C(=O)O)N(CCN(CCN(CC(=O)O)CC(=O)O)CC(=O)O)CC(=O)O]

58. The process according to claim 47, wherein Z′ is chosen from a lipid-lipid non-covalent association, —NH(O═)C—, —NHCH₂—, —SH₂C—, —C(═O)HN—,

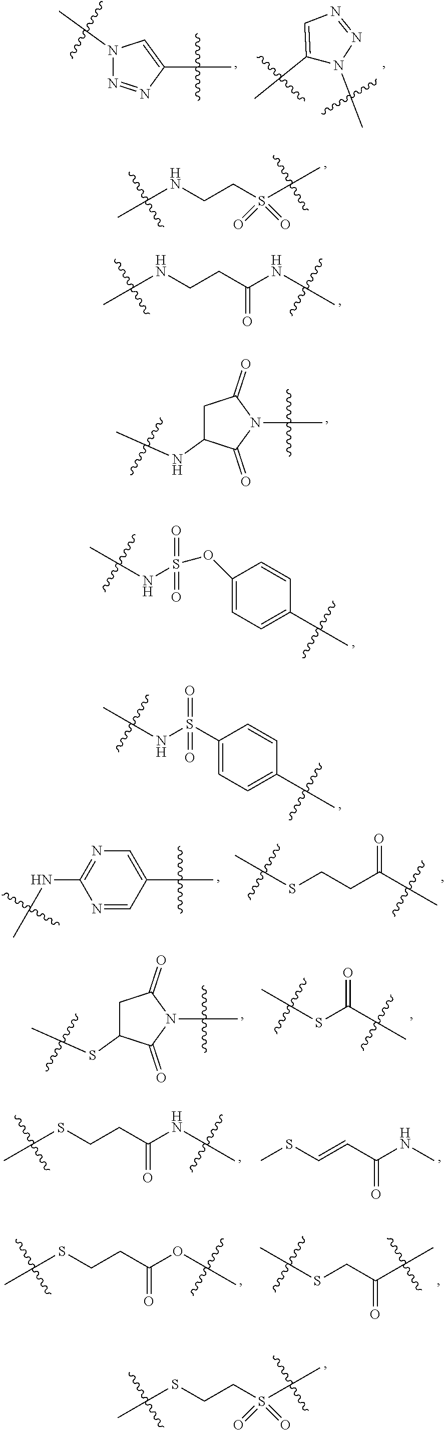

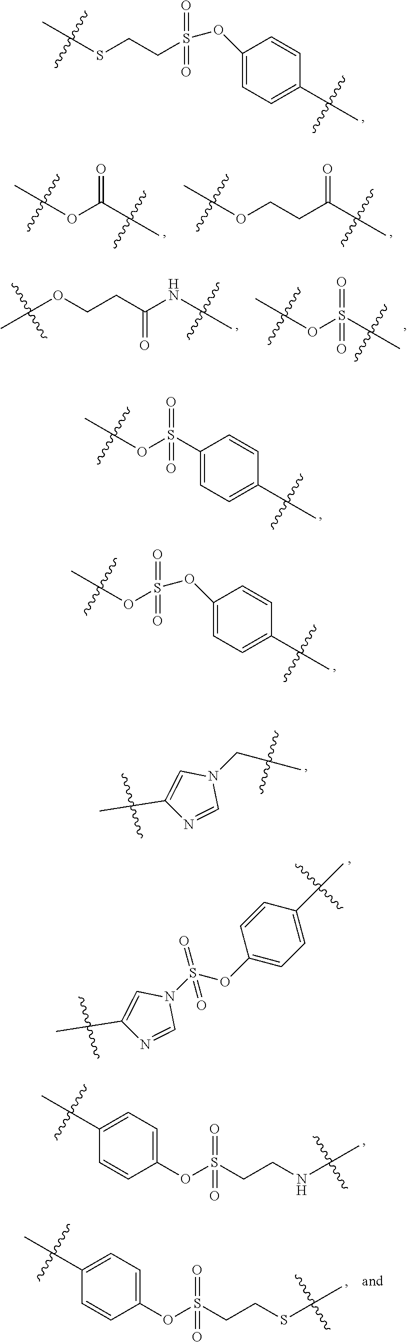

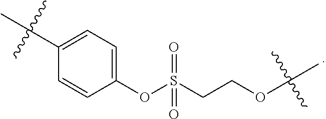 [SMILES: *OCCS(=O)(=O)Oc1ccc(*)cc1]

59. At least one entity chosen from compounds of Formula (II): 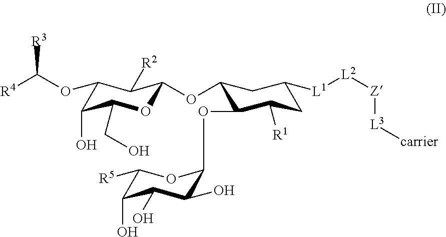
and pharmaceutically acceptable salts thereof, wherein
R¹ is chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, 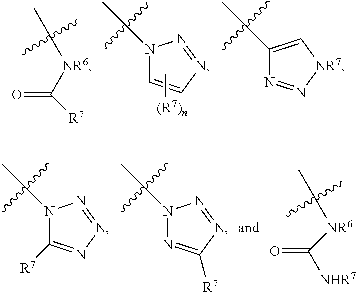
groups, wherein n is chosen from integers ranging from 0 to 2, R⁶ is chosen from H, C₁₋₁₂ alkyl, C₂₋₁₂ alkenyl, C₂₋₁₂ alkynyl, C₄₋₁₆ cycloalkylalkyl, and —C(═O)R⁷ groups, and each R⁷ is independently chosen from H, C₁₋₁₂ alkyl, C₂₋₁₂ alkenyl, C₂₋₁₂ alkynyl, C₄₋₁₆ cycloalkylalkyl, C₆₋₁₈ aryl, and C₁₋₁₃ heteroaryl groups;
R² is chosen from —OH, —OY¹, halo, —NH₂, —NHY¹, —NY¹Y², —OC(═O)Y¹, —NHC(═O)Y¹, —NHC(═O)NHY¹, and —NHC(═O)NY¹Y² groups, wherein Y¹ and Y², which may be the same or different, are independently chosen from C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₄₋₁₆ cycloalkylalkyl, C₂-12 heterocyclyl, C₆₋₁₈ aryl, and C₁₋₁₃ heteroaryl groups, or Y¹ and Y² may join together along with the heteroatom to which they are attached to form an optionally substituted, saturated or unsaturated ring;
R³ is chosen from —CN, —CH₂CN, —C(═O)Y³, —C(═O)OH, —C(═O)OY³, —C(═O)NH₂, —C(═O)NHOH, —C(═O)NHOCH₃, —NHCN, —C(═O)NHY³, —C(═O)NY³Y⁴, —S(═O)₂Y³, —S(═O)₂₀Y³, —S(═O)₂NH₂, —S(═O)₂NHY³, and —S(═O)₂NY³Y⁴ groups, wherein Y³ and Y⁴, which may be identical or different, are independently chosen from C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₁₋₁₃ heterocyclyl, and C₇₋₁₂ arylalkyl groups, or Y³ and Y⁴ join together along with the heteroatom to which they are attached to form an optionally substituted, saturated or unsaturated ring;
R⁴ is chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₁₋₁₂ alkoxy, C₄₋₁₆ cycloalkylalkyl, C₆₋₁₅ aryl, and C₂₋₁₃ heteroaryl groups;
R⁵ is chosen from —CN, C₁₋₁₂ alkyl, and C₁₋₁₂ haloalkyl groups;
L¹ is chosen from 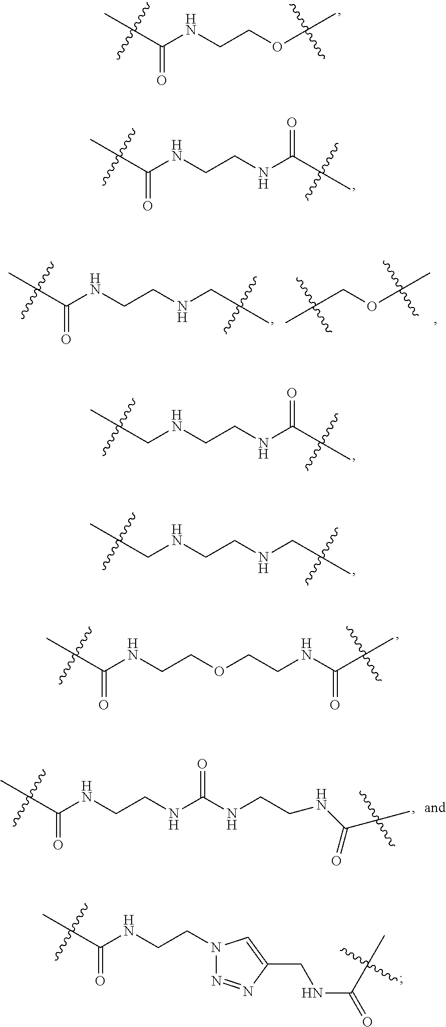
L² is chosen from —CH₂(OCH₂CH₂)ₘV—, —CH₂CH₂(OCH₂CH₂)ₘV—, and —(CH₂) mV— groups, wherein V is chosen from a bond, —O—, —OCH₂—, —NHC(═O)CH₂CH₂—, and —C(═O)NHCH₂CH₂—, and wherein m is chosen from integers ranging from 1 to 46;
L³ is chosen from a bond and linker groups; and
Z′ is chosen from a lipid-lipid non-covalent association, —C(═O)HN—, —CH₂NH—, —CH₂S—,

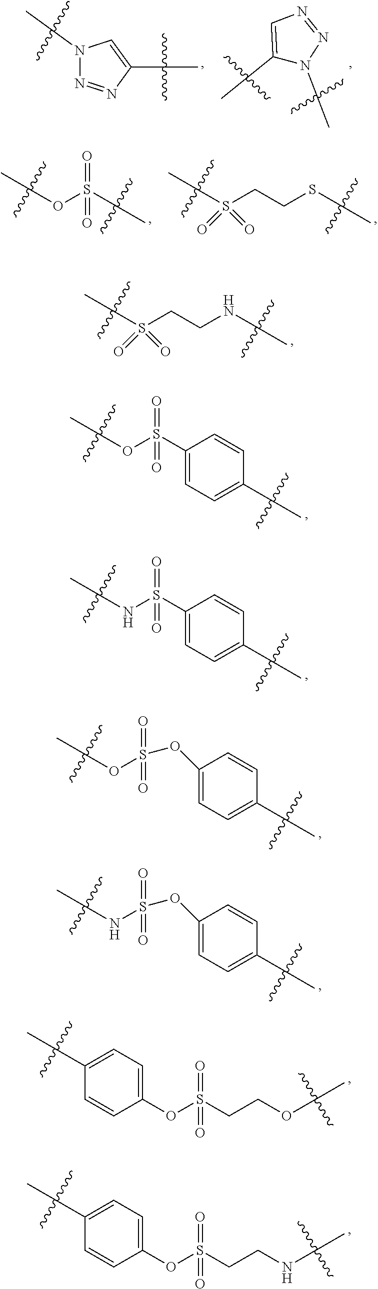

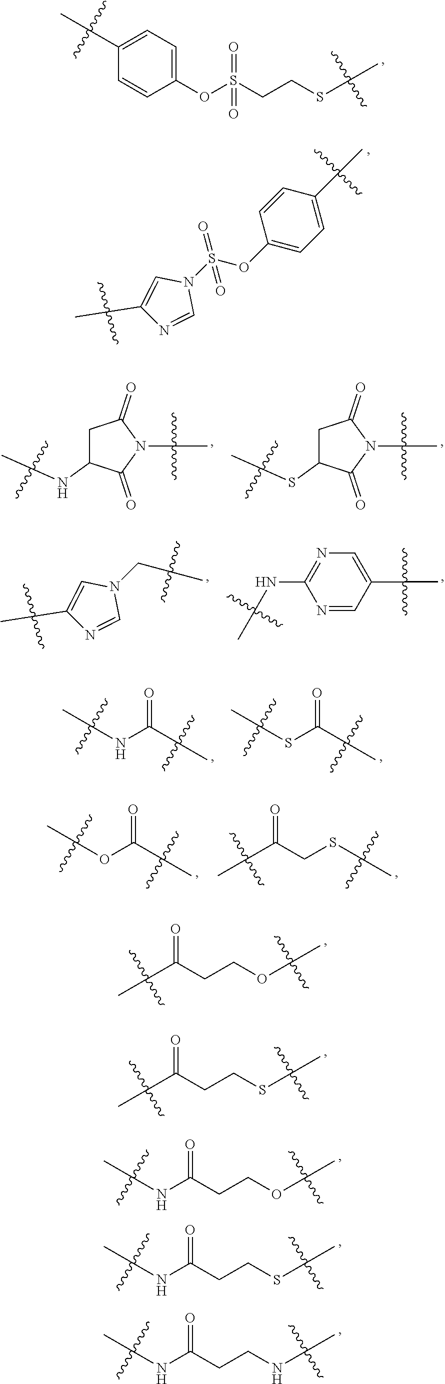

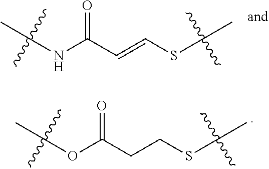 [SMILES: *NC(=O)/C=C/S* ; *OC(=O)CCS*]

60. A method for treatment and/or prevention of at least one disease, disorder, or condition, said method comprises administering to a subject in need thereof an effective amount of at least one entity of claim 59 or a composition comprising the same, wherein at least one therapeutic agent is directly or indirectly linked to or associated with said at least one entity of claim 59.

61. A method for treatment and/or prevention of at least one disease, disorder, or condition, said method comprises administering to a subject in need thereof an effective amount of at least one entity of claim 59 or a composition comprising the same, wherein at least one radiopharmaceutical is directly or indirectly linked to or associated with said at least one entity of claim 59.

62. The method of claim 61, wherein the at least one radiopharmaceutical is chosen from ¹⁷⁷Lu and ¹¹¹In.

63. The method of claim 60, wherein the at least one disease, disorder, or condition is chosen from cancers, inflammatory diseases, metastasis, and angiogenesis.

64. A method for diagnosing at least one disease, disorder, or condition, said method comprises administering to a subject in need thereof an effective amount of at least one entity of claim 59 or a composition comprising the same, wherein at least one therapeutic agent is directly or indirectly linked to or associated with said at least one entity of claim 59.

65. A method for diagnosing or imaging E-selectin expressing tissues, said method comprises administering to a subject in need thereof an effective amount of at least one entity of claim 59 or a composition comprising the same, wherein at least one diagnostic agent or at least one imaging agent is directly or indirectly linked to or associated with said at least one entity of claim 59.

66. The method of claim 65, wherein the at least one imaging agent is chosen from Gd³⁺, technetium 99 m, and cross-linked iron oxide superparamagnetic nanoparticles.
